SEQUENCE LISTING (27 sequences)

SEQ ID NO: 1 - DNA, 34 bp, Artificial sequence
  CI-f top strand primer
aatgtgtaatatttttatggtttttttagaatgg

SEQ ID NO: 2 - DNA, 31 bp, Artificial sequence
  CI-f top strand primer
ttacaaaaaaaatacctcttccttactaaac

SEQ ID NO: 3 - DNA, 26 bp, Artificial sequence
  CI-f bottom strand primer
tggtatttggaaagattgtaggaaag

SEQ ID NO: 4 - DNA, 24 bp, Artificial sequence
  CI-f bottom strand primer
caatacctctataacccttccaaa

SEQ ID NO: 5 - DNA, 24 bp, Artificial sequence
  CI-f top strand Ms-SNuPE primer
aataattttgtttttttggatatt

SEQ ID NO: 6 - DNA, 22 bp, Artificial sequence
  CI-f top strand Ms-SNuPE primer
aaattttgtttttggttgtaaa

SEQ ID NO: 7 - DNA, 21 bp, Artificial sequence
  CI-f bottom strand Ms-SNuPE primer
aggaatagaatttgagatatt

SEQ ID NO: 8 - DNA, 25 bp, Artificial sequence
  CI-f bottom strand Ms-SNuPE primer
taaattgttttaattagattaataa

SEQ ID NO: 9 - DNA, 19 bp, Artificial sequence
  CI-f site 1 Ms-SNuPE primer
tatggtttttttagaatgg

SEQ ID NO: 10 - DNA, 24 bp, Artificial sequence
  CI-f site 2 Ms-SNuPE primer
aataattttgtttttttggatatt

SEQ ID NO: 11 - DNA, 22 bp, Artificial sequence
  CI-f site 3 Ms-SNuPE primer
aaattttgtttttggttgtaaa

SEQ ID NO: 12 - DNA, 25 bp, Artificial sequence
  CI-f site 4 Ms-SNuPE primer
ttgttgttatgtgtaattatttttt

SEQ ID NO: 13 - DNA, 13 bp, Artificial sequence
  AP-PCR primer
cacatggttctgc

SEQ ID NO: 14 - DNA, 13 bp, Artificial sequence
  AP-PCR primer
gtctctatgaccc

SEQ ID NO: 15 - DNA, 13 bp, Artificial sequence
  AP-PCR primer
cttactgtgccac

SEQ ID NO: 16 - DNA, 31 bp, Artificial sequence
  CI-f bottom strand primer
gagtaaagatagaataaattgttttaattag

SEQ ID NO: 17 - DNA, 21 bp, Artificial sequence
  CII-d 5′-primer
gtttataggtttagaggtttt

SEQ ID NO: 18 - DNA, 25 bp, Artificial sequence
  CII-d 3′-primer
aacacataaacctattttaaactta

SEQ ID NO: 19 - DNA, 25 bp, Artificial sequence
  “A-Repeats” 5′-primer
tgatttatttattagaggttttagg

SEQ ID NO: 20 - DNA, 21 bp, Artificial sequence
  “A-Repeats” 3′-primer
acataaaaaaacaaactaccc

SEQ ID NO: 21 - DNA, 21 bp, Artificial sequence
  CII-d site 1 primer
ttttatttattgttattatgg

SEQ ID NO: 22 - DNA, 16 bp, Artificial sequence
  CII-d site 2 primer
ggtatagtttgagtat

SEQ ID NO: 23 - DNA, 26 bp, Artificial sequence
  CII-d site 3 primer
tattttttaatagtattattttttat

SEQ ID NO: 24 - DNA, 21 bp, Artificial sequence
  CII-d site 1 sequencing primer
ttttatttattgttattatgg

SEQ ID NO: 25 - DNA, 16 bp, Artificial sequence
  CII-d site 2 sequencing primer
ggtatagtttgagtat

SEQ ID NO: 26 - DNA, 24 bp, Artificial sequence
  CII-d site 3 sequencing primer
ttttttaataaggttatatttttt

SEQ ID NO: 27 - DNA, 26 bp, Artificial sequence
  CII-d site 4 sequencing primer
tattttttaatagtattattttttat

(19) United States
(12) Patent Application Publication
(10) Pub. No.: US 2004/0072197 A1
(43) Pub. Date: Apr. 15, 2004

(54) Assay for the detection and quantitation of hemimethylation

(76) Inventors: Peter A. Jones, La Canada, CA (US); Gangning Liang, Rowland Heights, CA (US); Yoshitaka Tomigahara, Toyonaka (JP); Peter W. Laird, South Pasadena, CA (US)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/416,075

(22) PCT Filed: Nov. 8, 2001
(86) PCT No.: PCT/US01/47141

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

Abstract There is disclosed a method to measure the fraction of DNA molecules that ishemi-methylated at a specific CpG dinucleotide in a particular DNA sequence, in a pool of DNA molecules having mixed DNA methylation states at said CpG dinucleotide. There is also disclosed methods to calculate the rates of de novo-, and maintenance-methylation per cell division that occur at said CpG dinucleotide. Additionally, there is disclosed methylation kits for use in measuring and quantitating hemi-methylation, and for calculating the rates of de novo-, and maintenance-methylation.

### CROSS-REFERENCE TO RELATED APPLICATIONS

This patent application claims the benefit of priority to U.S. Provisional Patent Application Serial No. 60/247,191, entitled “A NEW ASSAY FOR THE DETECTION AND QUANTITATION OF HEMIMETHYLATION,” filed on Nov. 8, 2000.

### TECHNICAL FIELD OF THE INVENTION

The present invention relates to DNA methylation, and in particular hemi-methylation. The present invention provides a method to measure the fraction of DNA molecules that is hemi-methylated at a specific CpG dinucleotide in a particular DNA sequence, in a pool of DNA molecules having mixed DNA methylation states at said CpG dinucleotide. The present invention also provides for methods to calculate the rates of de novo-, and maintenance-methylation per cell division that occur at said CpG dinucleotide.

### BACKGROUND OF THE INVENTION

The identification of early genetic changes in tumorigenesis is a primary focus in molecular cancer research. Characterization of the nature and pattern of cancer-associated genetic alterations will allow for early detection, diagnosis and treatment of cancer. Such genetic alterations in vertebrates fall generally into one of three categories: gain or loss of genetic material; mutation of genetic material; or methylation at cytosine residues in CpG dinucleotides within “CpG islands.” Among these, DNA methylation is uniquely reversible, and changes in methylation state are known to affect gene expression (e.g., transcriptional initiation of genes where CpG islands located at or near the promoter region) or genomic stability.

In higher order eukaryotic organisms, DNA is methylated only at cytosines located 5′ to guanosine in the CpG dinucleotide. This modification has important regulatory effects on gene expression predominantly when it involves CpG rich areas (CpG islands) located in the promoter region of a gene sequence. Extensive methylation of CpG islands has been associated with transcriptional inactivation of selected imprinted genes and genes on the inactive X chromosome of females. Aberrant methylation of normally unmethylated CpG islands has been described as a frequent event in immortalized and transformed cells and has been frequently associated with transcriptional inactivation of tumor suppressor genes in human cancers.

DNA methylases transfer methyl groups from a universal methyl donor, such as S-adenosyl methionine, to specific sites on the DNA. Mammalian cells possess methylases that methylate cytosine residues on DNA that are 5′ neighbors of guanine in CpG dinucleotides (CpG). This methylation may play a role in gene inactivation, cell differentiation, tumorigenesis, X-chromosome inactivation, and genomic imprinting. CpG islands remain unmethylated in normal cells, except during X-chromosome inactivation and parental specific imprinting where methylation of 5′ regulatory regions can lead to transcriptional repression. DNA methylation is also a mechanism for modifying the base sequence of DNA without altering its coding function. DNA methylation is a heritable, reversible and epigenetic change. Yet, DNA methylation has the potential to alter gene expression, which has profound developmental and genetic consequences.

DNA methylation is required for mammalian development (Li et al., 1992; Okano et al., 1999) yet the mechanisms responsible for the establishment and copying of methylation patterns remain almost completely unknown. 5-Methylcytosine is asymmetrically distributed in the genome and is most commonly found in CpG-poor regions, since most CpG islands in somatic cells remain methylation-free, except for the promoters of imprinted genes and genes on the inactive X-chromosome (Bird et al., 1985).

Methylation occurs after cytosine has been incorporated into DNA in a process catalyzed by DNA methyltransferases (“Dnmts”)which transfer the methyl group from S-adenosylmethionine to the 5′-position of the pyrimidine ring in, characteristically but not exclusively, the context of the palindromic CpG dinucleotide (Ramsahoye et al., 2000). Three Dnmt enzymes are known in mouse and human, and these have overlapping yet distinct abilities to methylate “hemimethylated” and completely unmethylated CpG dinucleotide pairs. Hemi-methylation is defined as a state in which the two opposing cytosines on either DNA strand in a single palindromic CpG dinucleotide differ in that one is methylated at the C-5 position, and the other is not.

The predominant Dnmt in the cell, Dnmt1, was cloned and characterized by Bestor and colleagues (1988) and is localized to replication machines in the S-phase nucleus (Leonhardt et al., 1992; Rountree et al., 2000). Since Dnmt1 shows a preference for hemimethylated CpG pairs (Gruenbaum et al., 1981; Bestor and Ingram, 1983), it is considered to be an excellent candidate for copying the pattern of methylation present on the parental strand after DNA has been replicated. However, Dnmt1 is capable of modifying unmethylated DNA in the test tube, and is thus also a candidate for inducing de novo methylation. The recently discovered Dnmts, Dnmt3a and 3b (Okano et al., 1998) show equal activities in vitro for unmethylated and hemimethylated substrates (Okano et al., 1998), and have been shown to be capable of de novo methylation of transfected DNA in culture (Hsieh, 1999) and in Drosophila (Lyko et al., 1999). Interestingly, satellite DNAs appear to be a preferred target for the human DNMT3B enzyme, because these satellite DNA sequences are specifically undermethylated in patients with ICF syndrome, characterized by germ-line mutations in the DNMT3B gene (Hansen et al., 1999; Okano et al., 1999; Xu et al., 1999).

Unfortunately, despite the identification of the above-mentioned Dnmts, and while DNA methylation has been implicated in gene regulation and cancer, especially in the context CpG islands, little is known about the significance, in these processes, of hemimethylation at specific CpG sites, or of the general extent of hemimethylation in CpG-poor DNA regions. This knowledge will require methods to measure characteristics (e.g., extent, rate) of hemimethylation at specific CpG dinucleotides.

Therefore, there is a need in the art to determine the role of specific Dnmts in copying the methylation patterns of a CpG-poor region of DNA. Additionally, there is a need in the art for a method to measure the fraction of DNA molecules that are hemi-methylated at a specific CpG dinucleotide in a particular DNA sequence, in a pool of DNA molecules having mixed DNA methylation states at said CpG dinucleotide. Furthermore, there is a need in the art methods to calculate the rates of de novo-, and maintenance-methylation per cell division that occur at said CpG dinucleotide.

### SUMMARY OF THE INVENTION

The present invention provides a method to measure the fraction of DNA molecules that is hemi-methylated at a specific CpG dinucleotide in a pool of DNA with mixed DNA methylation states at that CpG dinucleotide, comprising: extracting DNA from the various cell types; digesting the DNA sample with an excess of a methylation-sensitive restriction endonuclease that cleaves according to a recognition sequence motif comprising a palindromic CpG methylation site, whereby the endonuclease cleaves the DNA at a specific recognition motif position if the methylation state of the respective palindromic CpG methylation site is un-methylated, but not hemi-methylated or fully-methylated (e.g., HpaII); treating the DNA with bisulfite; amplifying the treated DNA by PCR with primers flanking the specific recognition motif position (e.g., the HpaII site); and determining the degree of hemi-methylation by averaging results obtained by sequence analysis of individual cloned PCR products for the presence of C or T at the first position of the specific CpG dinucleotide. Preferably, analysis of individual cloned PCR products for the presence of C or T at the first position of the specific CpG dinucleotide was accomplished using a methylation-sensitive single nucleotide primer extension (Ms-SNuPE) assay (Gonzalgo and Jones, 1997).

According to the present invention, the average rates per cell division of de novo methylation and of maintenance methylation are dictated by just two measurable variables: S=the percent of cytosines within CpGs found to be unmethylated by bisulfite analysis of one of the two single DNA strands, with prior HpaII digestion; and P=the percent of cytosines within CpGs found to be methylated by bisulfite analysis of one of the two DNA strands, without prior HpaII digestion.

The present invention further provides a novel set of equations to calculate the rate of de novo methylation (n) and the rate of maintenance methylation (m) at a single CpG dinucleotide within a HpaII site.

The present invention further provides a method calculate the rate of de novo methylation (n) and the rate of maintenance methylation (m) at a single CpG dinucleotide within a HpaII site comprising: determining a value for a variable S and for a variable P, where S=the percent of cytosines within CpGs found to be unmethylated by bisulfite analysis of one of the two single DNA strands, with prior HpaII digestion, and where P=the percent of cytosines within CpGs found to be methylated by bisulfite analysis of one of the two DNA strands, without prior HpaII digestion; and calculating the percent of HpaII-resistant molecules with double-stranded (full) methylation (D) as: D=100−2S; determining the value of H=2SP/(S+D) and F=DP/(S+D) and U=100−H−F, based thereon, whereby the rate of de novo methylation (n) at this CpG dinucleotide is defined as the fraction of unmethylated post-synthesis CpGs that undergoes conversion to fully methylated CpG per cell division: n=1−(U/(U+H/2)), and whereby the rate of maintenance methylation (m) at this CpG dinucleotide is defined as the fraction of post-synthesis hemimethylated CpGs that undergoes conversion to fully methylated CpG per cell division: m=1−(H/(F+H/2)).

The present invention further provides a hemimethylation detection kit useful for measuring the fraction of DNA molecules that is hemi-methylated at a specific CpG dinucleotide in a pool of DNA with mixed DNA methylation states at that CpG.

The present invention further provides a hemimethylation detection kit useful for measuring the rate of de novo methylation (n) and the rate of maintenance methylation (m) at a single CpG dinucleotide within a palindromic CpG methylation site.

### BRIEF DESCRIPTION OF THE DRAWINGS

FIG. 1A shows the patterns of methylation in ES Cells. The methylation status of the four CpG sites in individual molecules of DNA (Fragment CI-f) comprising the 440 bp sequence were assessed by cloning of individual molecules followed by MS-SNuPE analysis of the four sites as indicated.

FIG. 1B shows the methylation patterns of individual DNA molecules within the three cell types examined according to the present invention, where N=the number of DNA molecules assessed.

FIG. 2 shows the detection and quantitation of hemi-methylation in individual cloned DNA molecules according to the present invention. The experimental approach used to detect hemi-methylation in the ES cells comprises precutting genomic DNA with HpaII, followed by bisulfite treatment, then cloning individual PCR products, and assessing the methylation status of, e.g., site 2 by MS-SNuPE analysis.

FIG. 3 shows the measured steady state methylation levels, methylation rates, and an overview of the procedure used to calculate de novo (n) and maintenance (m) methylation rates (expressed as the fraction of substrate molecules converted per cell division) at a single CpG dinucleotide within a HpaII site, starting with the measurement of two experimental variables (“P” and “S”).

FIG. 4A summarizes results from such pulse-chase experiments which show that DNA synthesized during a 1 hr pulse of BrdU in wild type M1/3A/3B cells was not methylated to its final level until 24-48 hr after synthesis.

FIG. 4B shows that the delayed methylation of DNA requires active DNA methyltransferase enzymes.

FIG. 5 shows the maturation of methylation pattern cells. The methylation status of individual DNA molecules was determined immediately after a 1 hr pulse with BrdU, or after a chase period (24 hr) in medium not containing BrdU.

FIGS. 6A,B and C illustrate the proposed model for the interactivity between DNA methyltransferase enzymes in ES cells.

FIG. 6A depicts how methylation patterns are maintained in wild-type cells containing all three DNA methyltransferase enzymes.

FIG. 6B indicates how methylation is proposed to occur in cells lacking both Dnmt3a and Dnmt3b enzymes.

FIG. 6C shows the situation in M3A/3B cells, which lack Dnmt1. A mosaic methylation pattern is present with patches of methylation and unmethylation and a substantial fraction of hemi-methylated sites.

FIG. 7A shows methylation sensitive fingerprints of M1/3A/3B, M3A/3B, and M1 ES cell DNA after enzyme digestion with each of RsaI, RsaI with HpaII, or RsaI with MspI for 16 h. Bands which appear to be hypomethylated only in lane 2 are indicated by solid arrowheads (Class I). Bands which appear to be hypomethylated in both lanes 2 and 3 are indicated by open arrowheads (Class II). Functional DNA methyltransferase genes are indicated by solid squares and inactivated genes are indicated by open squares. GCP1, 2, 4 are GC poor primers.

FIG. 7B shows a Southern blot analysis of genomic DNA from M1/3A/3B, M3A/3B, and M1 ES cell DNA using isolated AP-PCR fragment CI-f.

FIG. 7C shows a Ms-SNuPE analysis for fragment CII-d from M1/3A/3B, M3A/3B, and M1 ES cell DNA. Quantitative methylation analysis of three CpG sites in CII-d. The percent methylation represents the average of three site using the following equation on PhosphoImager quantitation (methylated)C/[(methylated)C+(unmethylated)T]×100. CpG sites are represented by tick marks; striped boxes represent repetitive sequences; arrows represent PCR primers.

FIG. 8 shows the sequence properties of hypomethylated fragments isolated from methylation sensitive fingerprints in ES cells (FIG. 7A CpG sites are represented by tick marks; striped boxes represent repetitive sequences; upward arrows represent HpaII sites. *Indicates bands that are not included in FIG. 7A.

FIG. 9 shows the patterns of Methylation in ES Cells at Different Regions. The methylation status of individual molecules of DNA from the CI-f (Class I), CII-d and A-repeat regions (Class II) were assessed by cloning of individual bisulfite converted molecules followed by Ms-SNuPE or automated sequencing. CpG sites are represented by tick marks. Methylation status of CpG dinucleotides: (black wedges/circles) methylated; (white wedge/circles) unmethylated. Horizontal rows of wedges/circles indicate individual molecules that were sequenced after PCR amplification and cloning of bisulfite-treated DNA. The striped region indicated repeat sequences.

FIG. 10A shows the distribution of methylation status at CI-f and CII-d regions, and an overview of the procedure used to calculate distribution of methylation status (fully methylated, hemimethylated, and fully unmethylated) at a single CpG dinucleotide within a HpaII site, starting with the measurement of two experimental variables (“T” and “S”). A description of each variable is given in the first column, along with the equation used to derive the calculated variables (see Experimental Procedures). The second column illustrates the subset of methylation states representing that variable (white box), as well as all the other methylation states being assessed in that particular step (gray box). The unmethylated CpGs are absent in the case of the S and D variables, since the HpaI digestion removes these molecules from consideration at this step. The data obtained for the three cell lines for the measured variables “T” and “S” are shown in the columns on the right as percentages, followed by the absolute numbers of molecules assessed. Control experiments demonstrated that the top and bottom strand are equal in their methylation levels and rates. On average, differences between the newly synthesized strand and daughter strand after DNA replication should be distributed equally between the top and bottom strand in a large population of cells.

FIG. 10B shows the distribution of methylation status of CI-f and CII-d. F, indicates fully methylated; H, hemimethylated; U, fully unmethylated.

FIG. 11 shows the recovery of methylation after 5-Aza-CdR treatment. The indicated cell types were treated for 24 h with 3×10⁻⁷ M 5-aza-CdR and medium changed and the cells propagated further. DNA was extracted at the times indicated after the drug was added to the cultures and the methylation status of the CI-f (black) and CII-d fragments (white) measured by Ms-SNuPE analysis as indicated. Results given are the average values of two separate experiments.

FIG. 12 shows the methylation kinetics of newly synthesized DNA at CI-f and CII-d. ES cells containing the genes for the indicated DNA methyltransferases were pulsed for 1 hr with BrdU. The pulse was then removed and fresh medium added to the cells during the chase period. The DNA was extracted from the cells at various times after the pulse began and immunoprecipitated to isolate the BrdU containing DNA. The methylation status of HpaII sites in CI-f and CII-d at the indicated time points were determined by quantitative MS-SNuPE analysis. The data represent the mean values in two to six experiments and the error bars indicate the standard deviations.

### DETAILED DESCRIPTION OF THE INVENTION

Definitions

“Sodium bisulfite treatment” refers to the art-recognized treatment of genomic DNA with sodium bisulfite, whereby unmethylated cytosines in the DNA are converted to uracil by deamination, and where methylated cytosine residues are conserved (Frommer et al., P.N.A.S. USA 89:1827-31, 1992). Subsequent PCR amplification replaces the uracil residues with thymines and the 5-methylcytosine residues with cytosines. “GC C ntent” refers, within a particular DNA sequence, to the [(number of C bases+number of G bases)/band length for each fragment].

“CpG Island” refers to a contiguous region of genomic DNA that satisfies the criteria of (1) having a frequency of CpG dinucleotides corresponding to an “Observed/Expected Ratio” >0.6), and (2) having a “GC Content” >0.5. CpG islands are typically, but not always, between about 0.2 to about 1 kb in length.

“HpaII” refers to the art-recognized restriction endonuclease having the palindromic “CCGG” (top strand): “GGCC” (bottom strand) recognition motif that comprises two CpG dinucleotide sequences (one on each DNA strand) that function as potential methylation sites.

“RsaI” refers to the art-recognized restriction endonuclease having the “GTAC” (top strand): “CATG” (bottom strand) recognition motif.

“Excess” of a methylation-sensitive restriction endonuclease used to digest DNA refers to an amount sufficient, under the reaction conditions, to bring about essentially complete digestion of DNA, before bisulfite treatment to ensure accuracy.

“Methylation state” refers to the presence or absence of 5-methylcytosine (“5-mCyt”) at one or a plurality of CpG dinucleotides within a DNA sequence. Methylation states at one or more particular palindromic CpG methylation sites (each having two CpG CpG dinucleotide sequences) within such a DNA sequence include “methylated,” “fully-methylated” and “hemimethylated.”

“Hemimethylation” refers to the methylation state of a palindromic CpG methylation site, where only a single cytosine in one of the two CpG dinucleotide sequences of the palindromic CpG methylation site is methylated (e.g., 5′-CC^{M}GG-3′ (top strand): 3′-GGCC-5′ (bottom strand)).

“Hypermethylation” refers to the methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

“Hypomethylation” refers to the methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

“De Novo methylation” refers to the conversion of unmethylated post-synthesis CpG dinucleotide sequences (within a palindromic CpG methylation site) to fully methylated CpG sequences.

“Maintenance methylation” refers to the conversion of post-synthesis hemi-methylated CpG dinucleotide sequences (within a palindromic CpG methylation site) to fully methylated CpG sequences.

“Methylati n assay” refers to any assay for determining the methylation state of a CpG dinucleotide within a sequence of DNA.

“MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

“MethyLight” refers to the art-recognized fluorescence-based real-time PCR technique described by Eads et al., Cancer Res. 59:2302-2306, 1999.

“Ms-SNuPE” (Methylation-sensitive Single Nucleotide Primer Extension) refers to the art-recognized assay described by Gonzalgo & Jones, Nucleic Acids Res. 25:2529-2531, 1997.

“MSP” (Methylation-specific PCR) refers to the art-recognized methylation assay described by Herman et al. Proc. Natl. Acad. Sci. USA 93:9821-9826, 1996, and by U.S. Pat. No. 5,786,146.

“COBRA” (Combined Bisulfite Restriction Analysis) refers to the art-recognized methylation assay described by Xiong & Laird, Nucleic Acids Res. 25:2532-2534, 1997.

“MCA” (Methylated CpG Island Amplification) refers to the methylation assay described by Toyota et al., Cancer Res. 59:2307-12, 1999, and in WO 00/26401A1.

“DGE” refers to art-recognized 2-dimensional gel electrophoresis (2-DGE) methods.

Overview

The present invention provides a novel and straightforward technique to quantitate hemimethylation levels. Prior to the development of this method, there had been no accurate way to determine hemimethylation levels at specific CpG dinucleotides in the genome. Additionally, according to the present invention, the steady-state levels of hemimethylation and full methylation at a CpG dinucleotide provide sufficient information to allow calculation of the rates of de novo methylation and of maintenance methylation. The present invention provides a unique set of equations that reflect this concept. This led to the discovery that cells containing only the Dnmt3a and 3b enzymes show significantly more maintenance methyltransferase activity at this site than de novo methyltransferase activity. This contrasts with the prior view of these enzymes as predominantly de novo methyltransferases, but supports our proposed concept of post-Dnmt1 repair maintenance methyltransferases.

Thus, the present invention provides provides a method to measure the fraction of DNA molecules that is hemi-methylated at a specific CpG dinucleotide in a particular DNA sequence, in a pool of DNA molecules having mixed DNA methylation states at said CpG dinucleotide. The present invention also provides for methods to calculate the rates of de novo-, and maintenance-methylation per cell division that occur at said CpG dinucleotide.

The region selected for study was part of the transcribed region of a gene expressed in mouse mammary and thymus tissue. It was representative of the situation in most of the mammalian genome in which CpG islands, frequently located in promoters, are methylation free and most of 5-methylcytosine is found in coding regions and repetitive elements (Bird et al., 1985; Yoder et al., 1997; Jones, 1999). Because of the complexities of dealing with at least three enzymes with overlapping activities, the patterns and kinetics of methylation in wild-type cells were compared with cells containing either a homozygous mutation of the Dnmt1 gene or with a double Dnmt3a/3b knockout. Pulse chase experiments showed that, contrary to current assumptions, DNA was not methylated to its final level immediately after synthesis, but rather that methylation occurred in two phases. The data are most consistent with a model whereby Dnmt1 acts soon after replication but leaves gaps of hemimethylated DNA which are then filled in by Dnmt3a and/or 3b, which therefore have roles as “methylation repair” enzymes in addition to their potential roles as de novo methyltransferases.

Experimental Procedures

ES cell lines. ES cell culture, transfection, and selection were carried out as described previously (Li et al., 1992). J1 (M1/3A/3B) is a wild-type ES cell line of inbred 129/Sv background (Li et al., 1992). The Dnmt1^{-/-}(M3A/3B), Dnmt3a^{-/-}(M1/3B), Dnmt3b^{-/-}(M1/3A) and [Dnmt3a^{-/-}, Dnmt3b^{-/-}](M1) ES cells were previously described by Okano et al., (1999).

Pulse-chase experiments. ES cells were seeded at 9×10⁵ cells per 60 mm dish with feeder cells 72 hr before being pulsed for 1 hr in 2.5 ml medium containing 1×10⁴ M BrdU (Sigma). The BrdU-containing medium was then removed, the cells washed once with regular medium at 37° C. then chased with 4 ml medium supplemented with 1×10⁻⁴ M thymidine (Sigma). For inhibition studies, the BrdU was also chased with 10⁻⁶ M 5-Aza-CdR (Sigma), 0.1 ng/ml Trichostatin (TSA) (Sigma), 5×10⁻⁶ M Aphidicolin (Sigma), or 50 ng/ml Colcemid (Sigma). Genomic DNA was obtained following lysis with 100 mM NaCl, 10 mM EDTA, 1% SDS, and 1 μg/ml Proteinase K and purified by phenol and chloroform extractions and ethanol precipitation. The yield and purity of the DNA was determined by optical density measurement and 300 μg of DNA digested with 3000 units of RsaI (Roche) for 16 hr at 37° C. Digested DNA was purified again by phenol and chloroform extractions, ethanol precipitated, and resuspended in 500 μl TE buffer pH 7.5 with 50 μl 10× immunoprecipitation buffer (sodium phosphate (pH 7.0)), 0.14 M NaCl, 0.05% Triton X-100). The samples were denatured at 95° C. for 5 min, cooled on ice for 2 min, and immediately mixed with 2.4 μl anti-BrdU antibody (25 μg/ml) per μg DNA (Becton Dickinson) (Rideout et al., 1994). After 30 min incubation at room temperature with mild rocking, 0.46 μl anti-mouse IgG rabbit (2.6 mg/ml) per μg DNA (Sigma) was added and incubation continued for another 30 min at room temperature. The precipitate was collected after 5 min centrifugation at 13,000 rpm in a cold microcentrifage, dissolved in 200 μl TE pH 7.5 and treated with Proteinase K at 50° C. for more than 12 hr. DNA was deproteinized by phenol and chloroform extractions and ethanol precipitated.

Ms-SNuPE. The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites. Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

Methylation-sensitive single nucleotide primer extension (Ms-SNuPE) assays. The mean cytosine methylation levels of CpG sites 2, 3 in the fragment were determined by treatment of DNA (2 μg) with sodium bisulfite according to Frommer et aL (1992). Methylation analysis was performed using the methylation-sensitive single nucleotide primer extension (Ms-SNuPE) assay (Gonzalgo and Jones, 1997).

For Examples 1-5below:

The sequences of the primers used for bisulfite-treated DNA amplification were as follows: Top strand: 5′ primer (5′-AATGTGTAATATTTTTATGGTTTTTTAGAATGG-3′) (SEQ ID NO:1), 3′ primer (5′-TTACAAAAAAAATACCTCTTCCTTACTAAAC-3′) (SEQ ID NO:2). Bottom strand: 5′ primer (5′-TGGTATTTGGAAAGATTGTAGGAAAG-3′) (SEQ ID NO:3) 3′ primer (5′-CAATACCTCTATAACCCTTCCAAA-3′) (SEQ ID NO:4). PCR reactions were performed in 25 μl total volume under the following conditions: 100-200 ng bisulfite treated DNA, 20 mM Tris-HCl (pH 7.5), 2.5 mM MgCl₂, 100 mM KCl, 0.5 μM, final concentration of each primer, 200 μM each of the four deoxynucleotide triphosphates, and 0.5 U Taq polymerase (Sigma) complexed with Taq polymerase antibody (Clontech). DNA was initially denatured at 94° C. for 5 min, 94° C. for 1 min, 56° C. for 50 sec, and 72° C. for 1 min for 35 cycles, with a final extension at 72° C. for 4 min. PCR products were gel purified with the Qiaquick Gel Extraction Kit (Qiagen), and the template was resuspended in 30 μl H₂O.

Ms-SNuPEprimers: for top strand site 2 (5′-AATAATTTTGTTTTTTGGATATT-3′) (SEQ ID NO:5), site 3 (5′-AAATTTTGTTTTTGGTTGTAAA-3′) (SEQ ID NO:6), for bottom strand site 2 (5′-AGGAATAGAATTTGAGATATT-3′) (SEQ ID NO:7), site 3 (5′-TAAATTGTTTTAATTAGATTAATAA-3′) (SEQ ID NO:8).

Ms-SNuPE reactions. Ms-SNuPE reactions were performed in 10 μl total volume under the following conditions: 4 μl Qiaquick product, 20 mM Tris-HCl (pH 7.5), 2.5 mM MgCl₂, 100 mM KCl, 0.5 μM final concentration of each primer, and 1 μCi of either [³²P]dCTP or [³²P]dTTP. Primer extension conditions were: 95° C. for 2 min, 50° C. for 2 min, 72° C. 1 min. The reactions were combined with 4 μl stop solution before being denatured at 95° C. for 5 min and loaded onto a 15% denaturing polyacrylamide gel (7M urea). Quantitation of methylation levels was performed on a Molecular Dynamics PhosphorImager.

For Examples 6-8, below:

The PCR primers were designed specifically to amplify only bisulfite converted DNA and control experiments showed non-amplification of unconverted DNA with these primers. Also, sequencing of PCR products after bisulfite treatment showed less than 1% residual Cs at not CpG sites indicating that the assays were valid for methylation status at CpG sites. The sequences of the primers used for bisulfite-treated DNA amplification were as follows:

CI-f (Top strand) 5′ primer, 5′-AATGTGTAATATTTTTATGGTTTTTTTAGAATGG-3′ (SEQ ID NO:1), 3′ primer, 5′-TTACAAAAAAATACCTCTTCCTTACTAAAC-3′ (SEQ ID NO:2).

CI-f. (Bottom strand) 5′ primer 5′-GAGTAAAGATAGAATAAATTGTTTTAATTAG-3′ (SEQ ID NO:16) and 3′ primer, 5′-CAATACCTCTATAACCCTTCCAAA-3′ (SEQ ID NO:4).

CIId. 5′ primer, 5′-GTTTATAGGTTTAGAGGTTTT-3′ (SEQ ID NO:17), 3′ primer, 5′-AACACATAAAC CTATTTTAAACTTA-3′ (SEQ ID NO:18).

A-Repeats. 5′ primer, 5′-TGATTTATATTAGAGGTTTTAGG-3′ (SEQ ID NO:19), 3′ primer, 3′-AC ATAAAAAAACAAACTACCC-3′ (SEQ ID NO:20).

PCR Conditions. CI-f: 95° C. for 2 min, 95° C. for 1 min, 56° C. for 50 sec, and 72° C. for 1 min for 35 cycles, with a final extension at 72° C. for 10 min. CII-d: 95° C. for 2 min, 94° C. for 1 min, 50° C. for 50 sec, and 72° C. for 1.5 min for 40 cycles, with a final extension at 72° C. for 10 min. A-repeats: 95° C. for 2 min, 94° C. for 1 min, 50° C. for 50 sec, and 72° C. for 1 min for 40 cycles, with a final extension at 72° C. for 10 min. PCR products were gel purified with the Qiaquick Gel Extraction Kit (Qiagen), and the template was resuspended in 30 μl H₂O.

Ms-SNuPE Primers. CI-f: 5′-AATAATTTGTTTTTTTGGATATT-3′ (SEQ ID NO:5) (HpaII site); CII-d: 5′-TTTTATTTATTGTTATTATGG-3′ (SEQ ID NO:21) (site 1), 5′-GGTATAGTTTGAGTAT-3′ (site 2) (SEQ ID NO:22), 5-TATTTTTTAATAGTATTATTTTTTAT-3′ (SEQ ID NO:23) (site 3, HpaII Site).

Ms-SNuPE reactions were performed in 10 μl total volume under the following conditions: 4 μl Qiaquick product, 20 mM Tris-HCl (pH 7.5), 2.5 mM MgCl₂, 100 mM KCl, 0.5 μM final concentration of each primer, and 1 μCi of either [³²P]dCTP or [³²P]dTTP. Primer extension conditions of CI-f and CII-d were: 95° C. for 1 min, 46° C. for 30 sec., 72° C. for 20 sec. The reactions were combined with 4 μl stop solution before being denatured at 95° C. for 5 min and loaded onto a 15% denaturing polyacrylamide gel (7M urea). Quantitation of methylation levels was performed on a Molecular Dynamics PhosphorImager.

Bisulfite genomic sequencing by Ms-SnuPE. Bisulfite Genomic Sequencing by Ms-SNuPE or Automated DNA Sequencer. Traditionally patterns are determined by the cloning of individual bisulfite treated molecules followed by DNA sequencing. It was more convenient to subject individually cloned molecules to Ms-SNuPE analysis to rapidly assess how the four sites were methylated (CI-f and CII-d). The PCR products from bisulfite converted DNA were ligated into pCRII cloning vector (Invitrogen, San Diego, Calif.). Individual plasmid clones were amplified by M13 primers (forward and backward). The PCR product was used to sequence by Ms-SNuPE. The condition is the same as above. The primers for sequence are for CI-f: site 1 (5′-TATGGTTTTTTTAGAATGG-3′) (SEQ ID NO:9), site 2 (5′-AATAATTTTGTTTTTTTOGATATT-3′) (SEQ ID NO:10), site 3 (5′-AAATTTTGTTTTTGGTTGTAAA-3′) (SEQ ID NO: 11), site 4 (5′-TTGTTGTTATGTGTAATTATTTTTT-3′) (SEQ ID NO:12); and for CII-d (4 sites):5′-TTTTATTTATTGTTATTATGG-3′ (SEQ ID NO:24), 5′-GGTATAGTTTGAGTAT-3′ (SEQ ID NO:25), 5′-TTTTTTAATAAGGTTATATTTTTT-3′ (SEQ ID NO:26), 5-TATTTTTTAATAGTATTATTTTTAT-3′ (SEQ ID NO:27).

A-repeats clones were sequenced by automated DNA sequencer at USC/Norris Comprehensive Cancer Center microchemical facility.

5-Aza-CdR Treatments. Cells were plated (2×10⁶ cells/60 mm dish) and treated 24 h later with 3×10⁻⁷ M 5-Aza-CdR (Sigma). The medium was changed 24 h after drug treatment and every subsequent day. DNA was isolated at the indicated days after treatment as described (Pfeifer, et al. P.N.A.S. USA 87:8252-8256, 1990).

Hemimethylation assay. The fact that the enzyme HpaII will not cut the sequence CCGG in either the fully or hemimethylated configuration followed by bisulfite treatment was used to determine whether unmethylated cytosine occurred in the context of a CpG sequence in a HpaII insensitive site. Two ˜4 μg DNA were digested by RsaI and HpaII (10 units per μg) for 16 hr at 37° C. and then HpaII added (10 units per μg) for another 2 hr. The digests were then analyzed for methylation at site 2 by cloning of individual PCR-amplified bisulfite treated molecules as described above.

Methylation Equations. The relative amounts of full methylation, hemimethylation, and lack of methylation of a CpG dinucleotide within a HpaII site can be derived from two measurable variables: S=the percent of unmethylated cytosines found by bisulfite analysis of one of the two single DNA strands, after HpaII digestion; and P=the percent of methylated cytosines found by bisulfite analysis of one of the two DNA strands, without prior HpaII digestion.

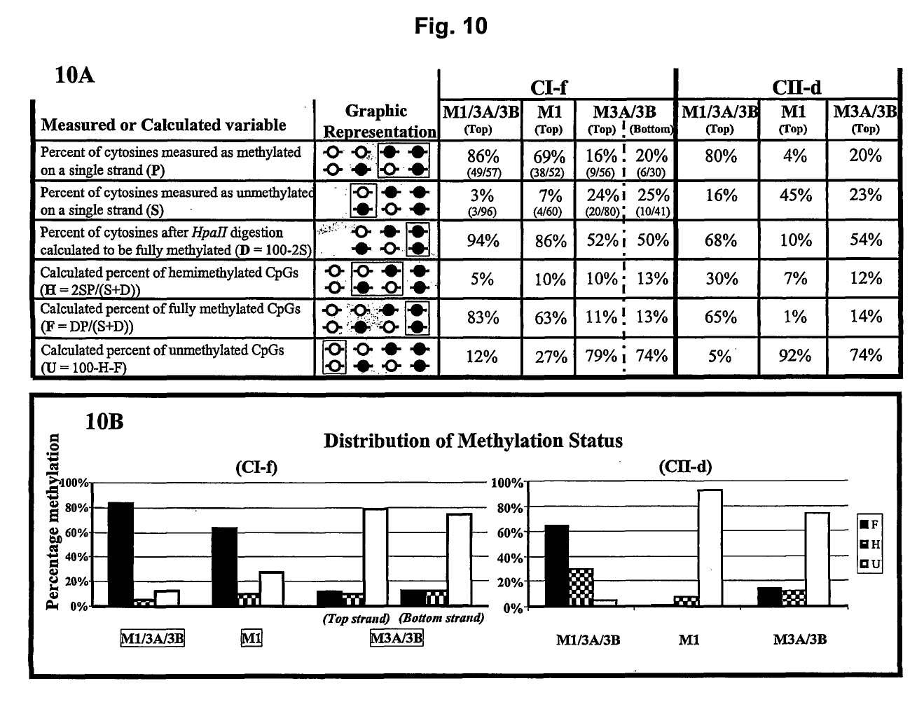

First, the percent of HpaII-resistant molecules with double-stranded (full) methylation (D) is calculated as: D=1-2S. In this equation, it is assumed that the top and bottom strands are equal in their methylation levels and rate (see FIG. 10 for additional support of this). On average, differences between the newly synthesized strand and daughter strand after DNA replication should be distributed equally between the top and bottom strand in a large population of cells.

The amount of hemimethylation (H) can be calculated as: H=2SP/(S+D) and full methylation (F) as: F=DP/(S+D). The basis for these two equations is the assumption that the ratio between hemimethylation and full methylation is constant for a particular DNA sample. Therefore, this ratio, which can be determined from the measurement of S (but which is lacking information on unmethylated DNA), can be applied to the measurement of all methylation within a single strand (P). Calculation of the percent unmethylated CpGs (U) can be calculated from H and F as: U=100−H−F.

Rate calculations. At equilibrium, the percent of DNA molecules that are unmethylated (U), hemimethylated on either strand (H), or fully methylated on both strands (F) at a CpG dinucleotide contained within a HpaII site, remains constant. Therefore, the relative ratios of these three methylation states resulting from each cell division as a consequence of the combined effects of de novo methylation and of maintenance methylation at this CpG should remain constant. If one assumes that de novo methylation of unmethylated post-synthesis CpGs results in full, double-stranded DNA methylation, then the average rates per cell division of de novo methylation and of maintenance methylation are dictated by just two measurable variables: S=the percent of cytosines within CpGs found to be unmethylated by bisulfite analysis of one of the two single DNA strands, with prior HpaII digestion; and P=the percent of cytosines within CpGs found to be methylated by bisulfite analysis of one of the two DNA strands, without prior HpaII digestion.

A novel set of equations was developed to calculate the rate of de novo methylation (n) and the rate of maintenance methylation (m) at a single CpG dinucleotide within a HpaII site, using just these two measured input variables. First, the percent of HpaII-resistant molecules with double-stranded (full) methylation (D) as: D=100−2S is calculated.

From this, H=2SP/(S+D) and F=DP/(S+D) and U=100−H−F can then be calculated. The rate of de novo methylation (n) at this CpG dinucleotide, is defined as the fraction of unmethylated post-synthesis CpGs that undergoes conversion to fully methylated CpG per cell division: n=1−(U/(U+H/2)). The rate of maintenance methylation (mn) at this CpG dinucleotide is defined as the fraction of post-synthesis hemimethylated CpGs that undergoes conversion to fully methylated CpG per cell division: m=1−(H/(F+H/2)).

Methylation-Sensitive AP-PCR. Methylation-sensitive AP-PCR and isolation of fragments of interest were performed as previously described Gonzalgo, et al., Cancer Research 57:594-599, 1997; Liang, et al., Genomics 53:260-268, 1998). The following CpG-poor primers were used for AP-PCR analysis: GCP1, 5′-CACATGGTTCTGC-3′ (SEQ ID NO:13); GCP2, 5′-GTCTCTATGACCC-3′ (SEQ ID NO: 14); GCP4, 5′-CTTACTGTGCCAC-3′ (SEQ ID NO:15). The following pairs were used in the AP-PCR reaction: GCP1/GCP2, GCP2/GCP4, GCP1/GCP4.

Southern Blot Analysis of DNA from Cell Lines. Ten μg of DNA from cell lines were separately digested with either 50 units of RsaI, 50 units each of RsaI and HpaII, or 50 units each of RsaI and MspI (Roche) at 37° C. for 16 hr. Digested genomic DNA was electrophoresed on 0.7% agarose gel and Southern transferred to Zeta-Probe (Bio-Rad) membranes overnight. Cloned DNA fragments that were previously isolated from AP-PCR polyacrylamide gels were subsequently used as probes for hybridization to these filters. Approximately 100 ng of plasmid insert DNA were ³²P-labeled by random priming and used to probe the filters. All hybridizations were performed in 500 m NaPO₄ (pH 6.8), 7% SDS, 1 mM EDTA (pH 8.0) at 65° C. for 16 hr. Membranes were washed twice at 65° C. with 2×SSC/1%SDS followed by three washes with 0.5×SSC/1% SDS at room temperature. They were then exposed to autoradiographic film at −80° C. Quantitation of methylation levels was performed on a Molecular Dynamics PhosphorImager.

Methylation Assay Procedures. Various methylation assay procedures are known in the art, and can be used in conjunction with the present invention for sequence determinations. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, use of methylation-sensitive restriction enzymes, etc.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

COBRA. COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the interested CpG islands, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

Preferably, assays such as “MethyLight” (a fluorescence-based real-time PCR technique) (Eads et al., Cancer Res. 59:2302-2306, 1999), Ms-SNuPE (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”;Toyota et al., Cancer Res. 59:2307-12, 1999) are used alone or in combination with other of these methods.

MethyLight. The MethyLight assay is a high-throughput quantitative methylation assay that utilizes fluorescence-based real-time PCR (TaqMan ®) technology that requires no further manipulations after the PCR step (Eads et al., Cancer Res. 59:2302-2306, 1999). Briefly, the MethyLight process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight may assay be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight-based kit) for MethyLight analysis may include, but are not limited to: PCR primers for specific gene (or methylation-altered DNA sequence or CpG island); TaqMan® probes; optimized PCR buffers and deoxynucleotides; and Taq polymerase.

Ms-SNuPE. The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites. Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

MSP. MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

MCA. The MCA technique is a method that can be used to screen for altered methylation patterns in genomic DNA, and to isolate specific sequences associated with these changes (Toyota et al., Cancer Res. 59:2307-12, 1999). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

It will be obvious to those skilled in the art that other art-recognized sequencing methods, such as direct sequencing, can be employed for purposes of the present invention, and that the above-described methylation assays are but examples of such methods that can be used.

Kits for Detection of Hemimethylated CpG-containing Nucleic Acid. The present invention provides a hemimethylation detection kit for measuring the fraction of DNA molecules that is hemi-methylated at a specific CpG dinucleotide in a pool of DNA with mixed DNA methylation states at that CpG. The present invention further provides a hemimethylation detection kit useful for measuring the rate of de novo methylation (n) and the rate of maintenance methylation (m) at a single CpG dinucleotide within a HpaII site.

The reagents required to perform one or more art-recognized methylation assays (including the Ms-SnuPE assay identified above) are combined with specific primers or probes to determine the hemimethylation state of CpG-containing nucleic acids, or the rate of de novo methylation (n) and the rate of maintenance methylation (m) at a single CpG dinucleotide within a HpaII site, according to the present invention.

For example, the Ms-SnuPE methylation assay could be used alone or in combination with other methylation assay methods known in the art, along with specific primers or probes to determine the hemimethylation state of a CpG dinucleotide within a particular genomic sequence.

### EXAMPLE1

### Methylation Patterns in ES Cells

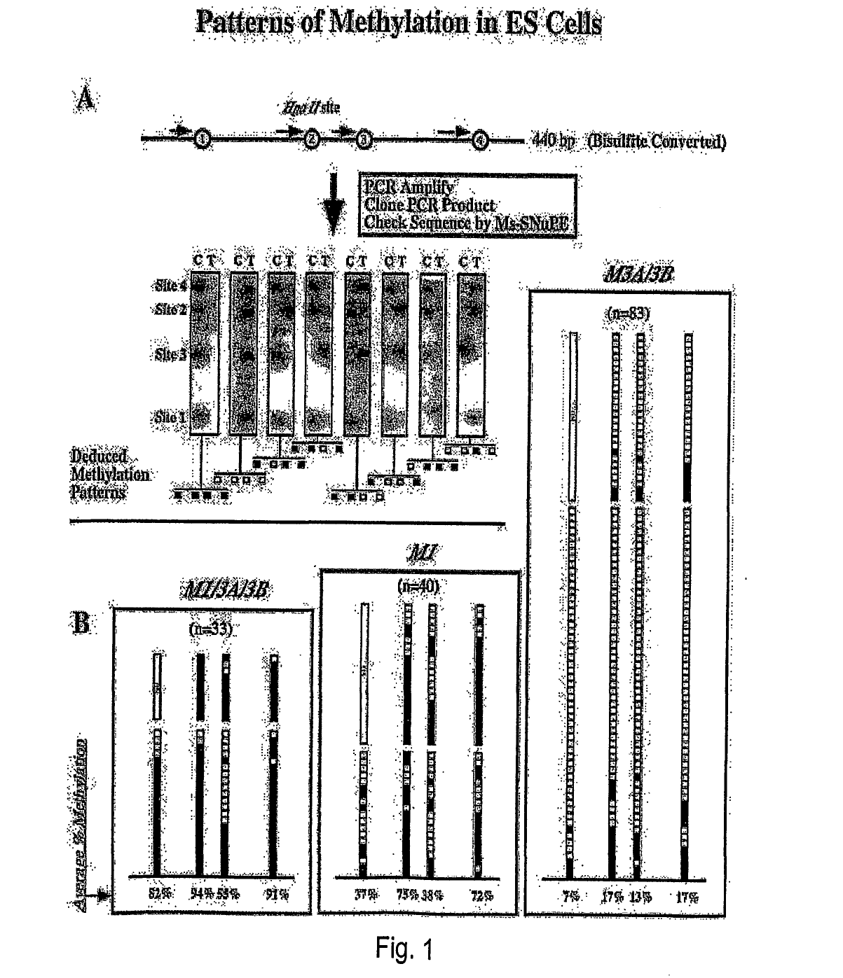

Detailed experiments were conducted on a 440-bp DNA fragment corresponding to a mouse EST (BE646975.1) (Fragment CI-f; GenBank accession number AK014019) expressed in mammary and thymus gland and therefore representative of the coding region of a typical mammalian gene in that it was both CpG poor and methylated. A single HpaII site and three additional CpG sites (FIG. 1A) which are residually methylated in the knockout ES cells were present in the fragment. The methylation status of the four CpG sites in individual DNA strands in cells containing all three Dnmts (M1/3A/3B cells) or containing Dnmt1 (M1 cells) or Dnmt3a and 3b only (M3A/3B cells) were assessed in detail to investigate potential differences in the patterns of methylation. The nomenclature used to describe the cell types was selected to focus on which enzymes were present in the cells rather than those which were absent. Minimal differences in methylation levels and patterns were seen in cells lacking either Dnmt 3a or 3b only.

Initially, the methylation patterns at sites 2-4 were determined by cloning of individual bisulfite treated molecules followed by DNA sequencing (top fragments in FIG. 1B). Typically however, the individually cloned molecules were subjected to Ms-SNuPE analysis to rapidly assess how the four sites were methylated (FIG. 1A).

FIG. 1A shows the patterns of methylation in ES Cells. The methylation status of the four CpG sites in individual molecules of DNA (Fragment Cl-f) comprising the 440 bp sequence were assessed by cloning of individual molecules followed by MS-SNuPE analysis of the four sites as indicated. Example gels of samples labeled with either dCTP “C” or dTTP “T” are shown. The presence of methylation at a given site is indicated by a band in the “C” lane of the Ms-SNuPE gel whereas a band in the “T” lane indicates no methylation. CpG sites in individual molecules are scored as being methylated (filled boxes) or unmethylated as shown (open boxes) so that patterns can be deduced.

Heterogeneous methylation patterns in M1/3A/3B cells and M1 cells were apparent with the four individual sites being methylated with differing efficiencies as indicated in FIG. 1B.

FIG. 1B shows the methylation patterns of individual DNA molecules within the three cell types examined according to the present invention, where N=the number of DNA molecules assessed. The data from the upper section within each panel were obtained by direct sequencing of fragments containing sites 2 through 4. The bottom sections are for all four sites. The status of methylation at the sites in individual molecules is read from left to right. FIG. 1B also shows the average percent of methylation at the four sites in the three cell types examined.

Sites 2 and 4 were most methylated on average whereas sites 1 and 3 were less efficient methyl acceptors. The methylation pattern in the M3A/3B cells were, however, completely different in that two classes of molecules were present. The majority of molecules in which all four sites were simultaneously assessed were methylation-free (72%), 14% had only one site methylated but 14% had multiple sites methylated on a single molecule.

This data was most consistent with Dnmt3a/3b being processive or co-operative enzymes, which tended to methylate sites sequentially although not with 100% efficiency, since sites 2 and 4 were more methylated than sites 1 and 3. The differential levels of methylation at the four sites made it difficult to distinguish between the possibilities that the enzymes act processively or rather that methylation of one site increases the probability of methylation of adjacent sites. ES cells containing Dnmt3a or 3b as the sole methyltransferase were not tested, and therefore it was difficult to effectively distinguish between any differential specificity between the enzymes. Analysis of the methylation status of the cytosines on the paired bottom strands revealed no strand bias in any of the cell types analyzed.

### EXAMPLE 2

### A New Assay for the Detection and Quantitation of Hemimethylation

Bisulfite sequencing has opened the door for the detailed analysis of methylation patterns (Frommer et al., 1992), yet it has not heretofore been possible to assess the degree of hemi-methylation (i.e., when only a single cytosine in the CpG palindrome is methylated), because the parental paired DNA strands are separated before analysis. Knowledge of the degree of hemi-methylation is crucial to understanding how patterns are copied.

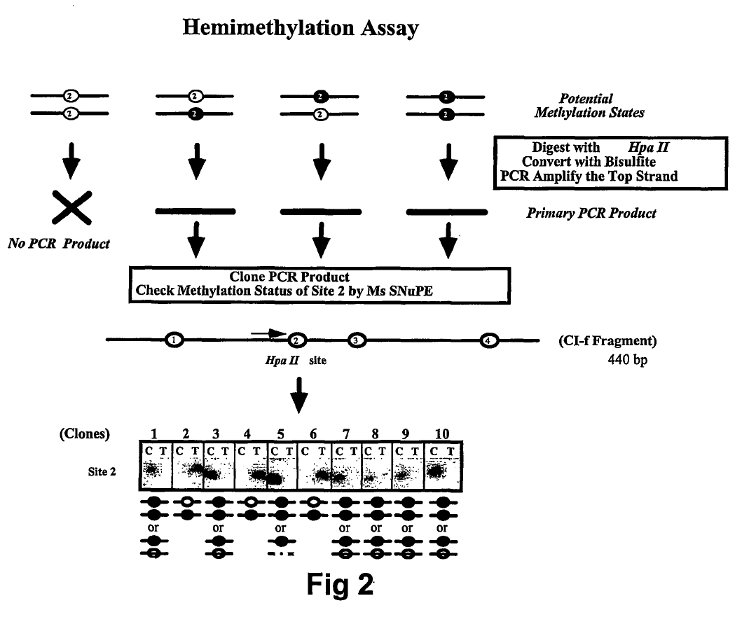

The fact that the enzyme HpaII will not cut the recognition motif sequence CCGG in either the fully or hemi-methylated configuration (Gruenbaum et al., 1981) followed by bisulfite treatment was used to determine whether unmethylated cytosine occurred in the context of a CpG sequence in a HpaII-resistant site (see FIG. 2).

FIG. 2 shows the detection and quantitation of hemi-methylation in individual cloned DNA molecules according to the present invention. The experimental approach used to detect hemi-methylation in the ES cells comprises precutting genomic DNA with HpaII, followed by bisulfite treatment, then cloning individual PCR products, and assessing the methylation status of, e.g., site 2 by MS-SNUPE analysis. Typical results are shown for these MS-SNuPE results in which a signal in the T lanes of example clones indicates hemi-methylation with the top strand being unmethylated. The presence of a signal in the “C” lane indicates that the site may be either hemi-methylated (i.e., the bottom strand is unmethylated) or fully methylated. The distribution between these two scenarios is determined by calculation, assuming that there is an equal probability of hemi-methylation of either the top or bottom strand.

Specifically, DNA extracted from the various cell types was digested with an excess of HpaII, bisulfite treated and then amplified by PCR with primers flanking the HpaII site (site 2). Since the HpaII site is located between the primers, molecules without fall or hemi-methylation of site 2 would not be amplified and thus be excluded from the analysis. The degree of hemi-methylation at a particular site can be accurately determined by averaging results obtained by analysis of individual cloned PCR products for the presence of C or T at site 2 (for example). Since only the top strand is analyzed in this approach and methylation of either the top or bottom strand is possible, the degree of hemi-methylation was calculated as indicated in “Experimental Procedures,” above. These calculations assume that there is an equal probability of hemi-methylation of either the top or bottom strand, a supposition which was supported by additional experiments (see Examples 6-8 below).

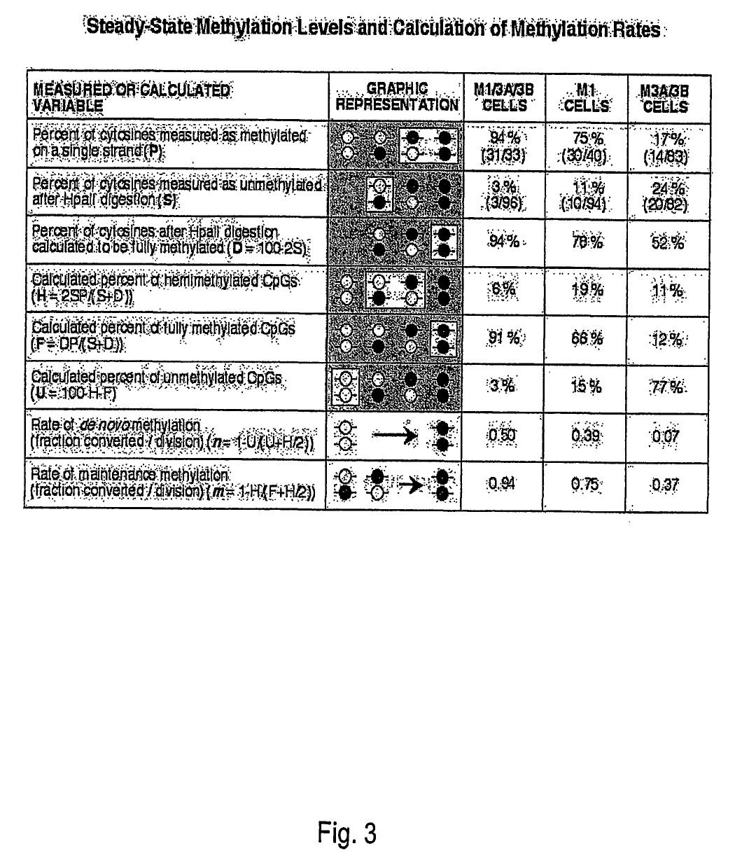

This approach allowed for the determination of the distribution of methylation of site 2 with respect to complete unmethylation, hemi-methylation and full methylation in the three different cell types (FIG. 3).

FIG. 3 shows the measured steady state methylation levels, methylation rates, and an overview of the procedure used to calculate de novo (ii) and maintenance (m) methylation rates (expressed as the fraction of substrate molecules converted per cell division) at a single CpG dinucleotide within a HpaII site, starting with the measurement of two experimental variables (“P” and “S”). A description of each variable is given in the first column, along with the equation used to derive the calculated variables (see “Experimental Procedures,” above). The second column illustrates the subset of methylation states representing that variable (white box), as well as all the other methylation states being assessed in that particular step (gray box). The unmethylated CpGs are absent in the case of the S and D variables, since the HpaII digestion removes these molecules from consideration at this step. The data obtained for the three cell lines for the measured variables “P” and “S” are shown in the columns on the right as percentages, followed by the absolute numbers of molecules assessed. It is assumed that the top and bottom strand are equal in their methylation levels and rates. On average, differences between the newly synthesized strand and daughter strand after DNA replication should be distributed equally between the top and bottom strand in a large population of cells.

As expected, the great majority of sites in wild type cells were fully methylated with only a minor proportion of hemimethylated sites (6%). The high percentage (19%) of hemi-methylated sites in M1 cells (i.e., containing Dnmt1 only), was not anticipated since it was assumed that this enzyme would be highly efficient as a hemi-methylase and would rapidly convert half-methylated sites to full methylation following DNA replication. Since there were fewer hemi-methylated sites in the wild-type cells containing all three enzymes (M1/3A/3B), the data indicate that Dnmt3a/3b are acting as efficient hemi-methylases in addition to their roles as de novo methylases.

FIG. 3 also shows the rates of de novo and maintenance methylation which could be calculated from this data (see “Experimental Procedures,” above). Interestingly, FIG. 3 shows that M3A/3B cells had higher rates of hemi-methylase activity (0.37) than of de novo methyltransferase activity (0.07). These results indicate that Dnmt1 and Dnmt3a/3b acted coordinately to ensure a low proportion of hemi-methylated sites in the wild type cells.

### EXAMPLE 3

### Kinetics of Methylation of Newly Synthesized DNA

The timing of methylation with respect to DNA synthesis was investigated. The demonstration that Dnmt1 is localized at the replication machinery in the S-phase nucleus (Leonhardt et al., 1992), and interacts with PCNA (Chuang et al., 1997) has led to the expectation that the methylation pattern is copied immediately after synthesis (Araujo et al., 1998), although earlier studies suggested that some methylation might be delayed so that cytosine modification occurred in a biphasic fashion (Adams, 1971; Woodcock et al., 1982, Woodcock et al., 1986).

A 1 hr BrdU pulse, followed by chasing for various times was used to investigate the timing of methylation with respect to synthesis. DNA containing BrdU was isolated by immunoprecipitation (Rideout et al., 1994) and the methylation status of sites 2 and 3 within the DNA fragment (see FIG. 1A for map) measured by quantitative Ms-SNuPE analysis.

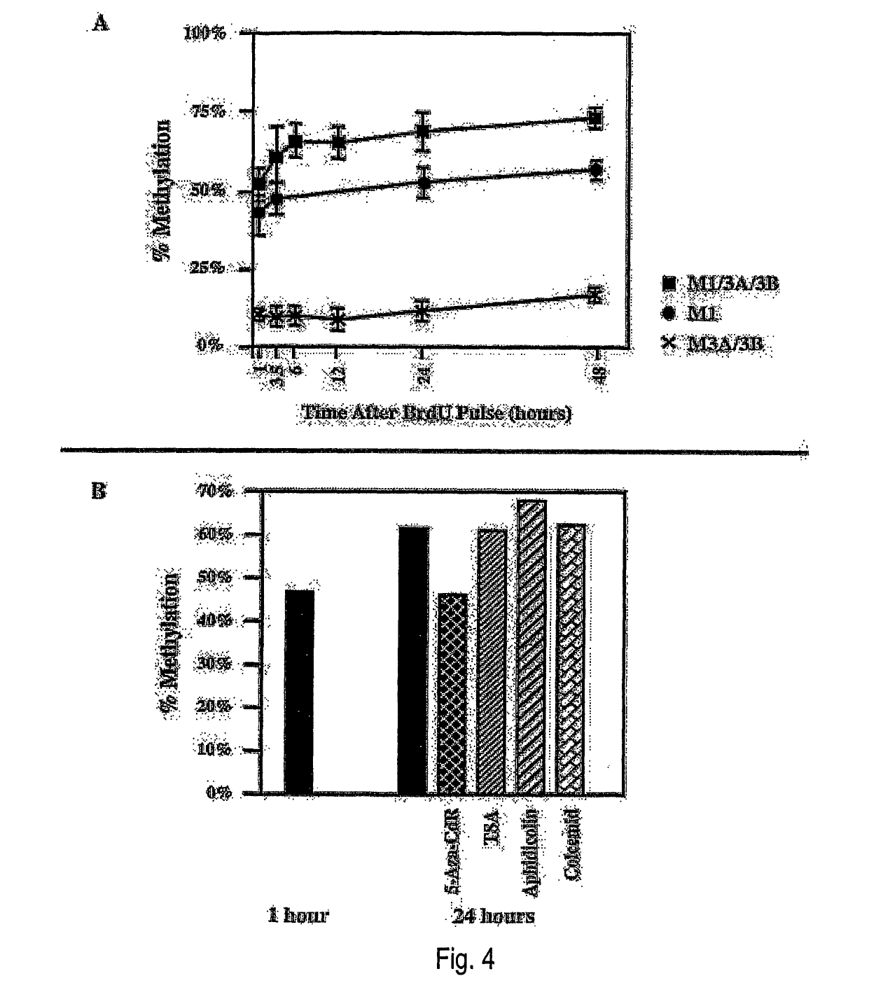

FIG. 4A summarizes results from such pulse-chase experiments which show that DNA synthesized during a 1 hr pulse of BrdU in wild type M1/3A/3B cells was not methylated to its final level until 24-48 hr after synthesis. ES cells containing the genes for the indicated DNA methyltransferases were pulsed for 1 hr with BrdU. The pulse was then removed and fresh medium added to the cells during the chase period. The DNA was extracted from the cells at various times after the pulse began and immunoprecipitated to isolate the BrdU containing DNA. The methylation status of sites 2 and 3 were averaged at the indicated time points after determination by quantitative MS-SNuPE analysis. The data represent the mean values for the determinations on the top and bottom strands in two to three experiments and the error bars indicate the standard deviations.

Some methylation was therefore delayed with respect to synthesis and was also seen in cells containing Dnmt1 in combination with either Dnmt3a or 3b, but was less marked in M1 cells (FIG. 4A). The methylation level of DNA synthesized in M3A/3B cells was significantly lower than that in the other cell types and showed little increase until 48 hr after synthesis. The data indicated that the post-synthesis methylation in the wild-type M1/3A/3B cells might be due to the activities of Dnmt3a and 3b, because the degree of post-synthesis methylation was less noticeable in the M1 cells, which lacked these two enzymes.

FIG. 4B shows that the delayed methylation of DNA requires active DNA methyltransferase enzymes. Cells containing all three DNA methyltransferase enzymes (M1/3A/3B) were pulsed for 1 hr with BrdU and DNA collected after 1 hr from some of the cultures. The medium in the remaining cultures was changed to medium containing the indicated compounds during the chase period and DNA collected 24 hr after the beginning of the experiment. BrdU-containing DNA was immunoprecipitated and the level of methylation in this DNA determined by quantitative MS-SNuPE analysis.

The delayed methylation on the newly synthesized DNA strand in the wild-type M1/3A/3B cells required the presence of active Dnmts, because treatment with 5-aza-2′-deoxycytidine at the time the BrdU pulse was removed resulted in no further methylation of the pulsed strand (FIG. 4B). Because the drug forms a covalent bond with Dnmts, it induces a rapid “transient knockout” of active enzymes (Taylor and Jones, 1982).

FIG. 4B also shows that the maturation process was not blocked by drugs which inhibit histone deacetylase (Trichostatin A, “TSA”), DNA synthesis (Aphidicolin) or mitosis (Colcemid).

Thus, active methyltransferases but not histone acetylation, DNA synthesis or mitosis were necessary for the delayed methylation to occur.

### EXAMPLE 4

### Patterns of Methylation of Newly Synthesized DNA

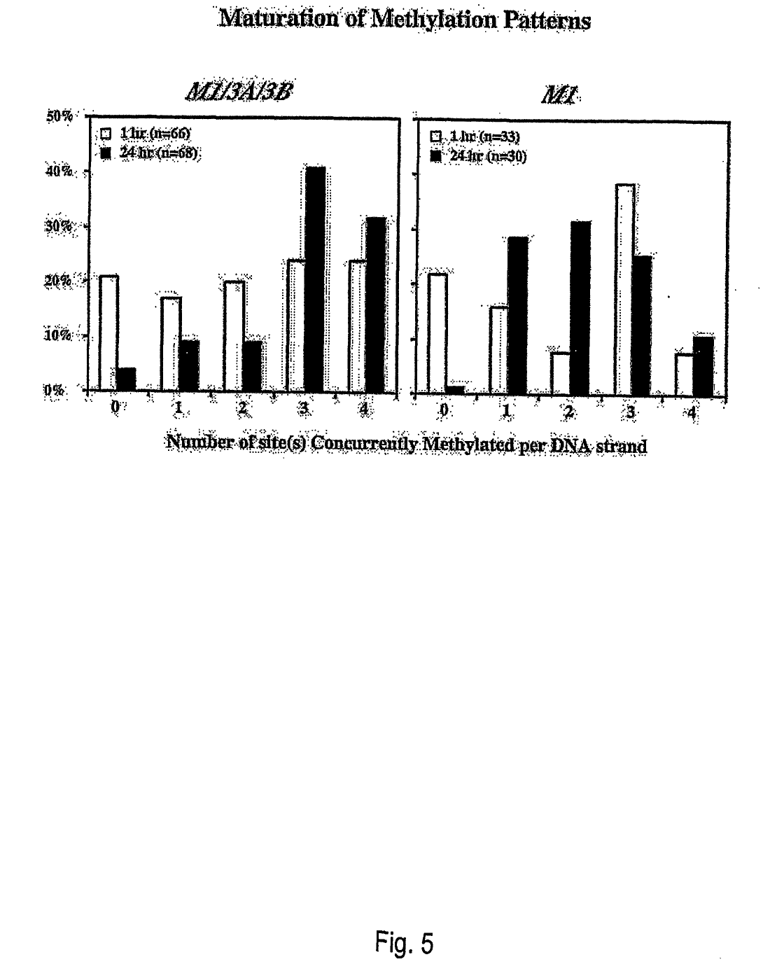

The above experiments yielded information of the average methylation levels of two of the four sites with respect to time after synthesis. Individual DNA strands collected at 1 hr and 24 hr after BrdU pulse and chase, respectively, were cloned to see how the patterns evolved on a molecule by molecule basis by plotting the number of sites concurrently methylated per strand at 1 hr and 24 hr (FIG. 5).

FIG. 5 shows the maturation of methylation pattern cells. The methylation status of individual DNA molecules was determined immediately after a 1 hr pulse with BrdU, or after a chase period (24 hr) in medium not containing BrdU. DNA collected from these two time points was immunoprecipitated to isolate BrdU-containing DNA, and the methylation status of individual molecules determined to assess the methylation density of these molecules as a function of time after synthesis as in FIG. 1. Data are given as the percentage of individual molecules containing between 0 and 4 sites concurrently methylated on an individual molecule.

Surprisingly, DNA collected immediately after the 1 hr pulse from M1/3A/3B cells, showed that a considerable proportion (38%) of molecules contained none or only one of the four sites methylated and a relatively even distribution of methylation densities. The pattern shifted substantially in the 24 hr chase period with most of the molecules now acquiring concurrent methylation at three or four sites. Newly synthesized DNA in M1 cells had 40% of molecules with zero or one site methylated, 9% having two sites and 50% having three or four sites methylated. Again, this pattern shifted during the chase period, but the mode of the density distribution was two sites methylated per molecule as compared to the three in M1/3A/3B cells.

Thus, newly synthesized DNA contained unmethylated “patches” in both cell types as manifest by the occurrence of molecules with all four sites simultaneously unmethylated which were very much reduced in the post-synthesis phase. This reduction was most likely achieved by Dnmt3a and/or 3b, in the wild type cells since a less striking change occurred in the M1 cells which lacked these enzymes.

The same pattern analysis was not performed on the M3A/3B cells, because the majority of molecules (72%) were unmethylated at equilibrium (see FIG. 1), making it technically burdensome to clone and assess a large enough number of them for meaningful interpretation.

### EXAMPLE 5

### Subcellular Localization of DNA Methyltransferases

If the discontinuity observed in the kinetics of post-synthetic restoration of DNA methylation levels was indeed attributable to distinct methyltransferase activities, with Dnmt1 mainly responsible for maintenance activity immediately following DNA synthesis and Dnmt3a and/or 3b being active later in the cell cycle, then Dnmt3a and/or 3b would not be expected to be as strongly associated with toroidal replication foci, as has been observed for Dnmt1.

The subcellular localization of the three methyltransferases was determined by the transfection of plasmids containing the three genes fused to green fluorescence protein (GFP) into mouse C3H 10T1/2 C18 cells. Cells were double stained with an antibody to PCNA labeled with Texas red and examined by confocal microscopy 48-72 hr after transfection. Cells transfected with DNMT-1 confirmed earlier studies that this protein often co-localized with PCNA, particularly in toroidal structures presumably representing replication machines at late S phase (Leonhardt et al., 1992; Chuang et al., 1997; Rountree et al., 2000).

Dnmt3a and 3b were localized almost entirely to the nucleus and were more diffusely present in nuclei containing these toroids. Interestingly, Dmnt3b, unlike Dnmt3a showed some co-localization with the PCNA-containing toroids.

Thus, Dnmt3b, but not Dmnt3a may associate with replication machines.

### EXAMPLE 6

### (Genome Scanning was Used to Identify Sequences to Investigate Methylation Patterns)

The findings of Examples 1-5 (above) were extended. Specifically, a genome scanning approach was used to investigate the patterns of methylation in the various knockout ES cells in CpG poor and CpG rich regions to determine the roles of the enzymes in carrying out the bulk of methylation in mouse embryonic stem (ES) cells.

The methylation levels of CpG-poor sequences were, in general, uniformly reduced in Dmnt1-deficient cells. However, there was considerable variability among different regions in the efficiency with which DNA methylation was retained in Dnmt3a/3b-deficient cells indicating a sequence preference of the. Dnmt1 enzyme.

MS AP-PCR was used to fingerprint the methylation patterns in a panel of ES cells containing different combinations of DNA methyltransferases (Dnmt1 (−/−) or Dnmt3a ((−/−)/3b (−/−)). The MS AP-PCR method allows for a methylation pattern to be easily obtained and relies on the differential susceptibilities of unmethylated and methylated CCGG sites to cutting by the enzyme HpaII giving a valid fingerprint of the methylation status of CpG islands. The purpose and advantage of Ms AP-PCR was to perform a rapid and global screen of genome which would then allow identification of representative sequences for more detailed analysis. We initially focused on CpG poor regions of DNA in the current work, since these are the regions of DNA in which the majority of 5-methylcytosine is found.

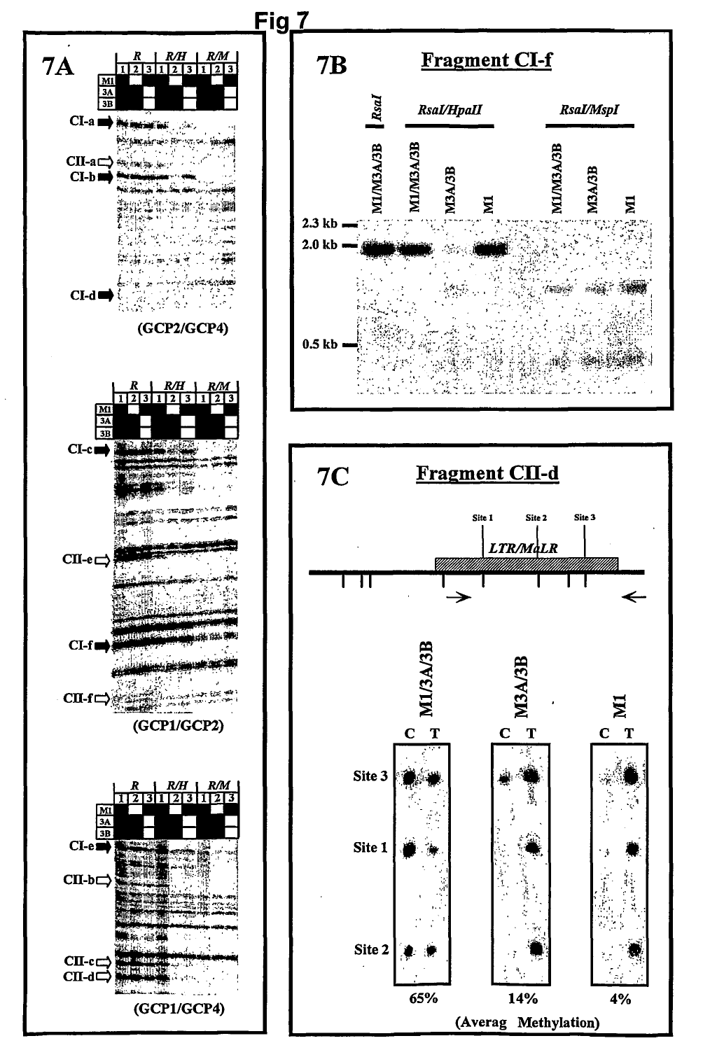

FIG. 7A shows an example of an analysis of DNA extracted from the various cell types using CpG poor primers to target regions of DNA not located in CpG islands. The nomenclature used for the cells was selected to highlight which gene products were active in the cells rather than those which were absent. Analysis of the fingerprints showed a uniform loss of methylation at most evaluable bands in cells which contained Dnmt3a and 3b only M3A/3B), as compared to the other cell types examined (see lanes 2 and bands indicated by filled in arrows). The decreased intensities in the Rsa/HpaII digests of DNA from these cells suggested a generalized decrease in methylation in cells without a functional Dnmt1 enzyme and a lack of marked sequence specificity of the retained Dnmt3a and 3b, since no bands were apparent which were completely methylated or unmethylated relative to the wild-type or mutant cell types.

An important distinction was observed in cells which contained Dnmt1 only (M1 cells, lane 3). Their methylation pattern was similar to that of the wild-type cells for many of the bands. However there were 6/12 evaluable bands present in which clearly decreased methylation was apparent (indicated by open arrows). This contrasted to the situation in the M3A/3B cells in which there was a generalized decrease in all sequences examined. Since the AP-PCR method is only semiquantitative, the level of methylation of some of the bands with decreased intensities on AP-PCR gels were assessed using either Southern blot analysis or Ms-SNuPE analysis (FIGS. 7B and 7C). Results confirmed the decreases apparent from the AP-PCR gels, thus validating the approach.

There were, therefore, two classes of fragments visible in the fingerprints. We defined Class I sequences as those that had decreased methylation in M3A/3B cells (i.e., lacking Dnmt1), but close to normal methylation levels in M1 cells. Class H sequences, on the other hand showed loss of methylation in both M1 and M3A/3B cells.

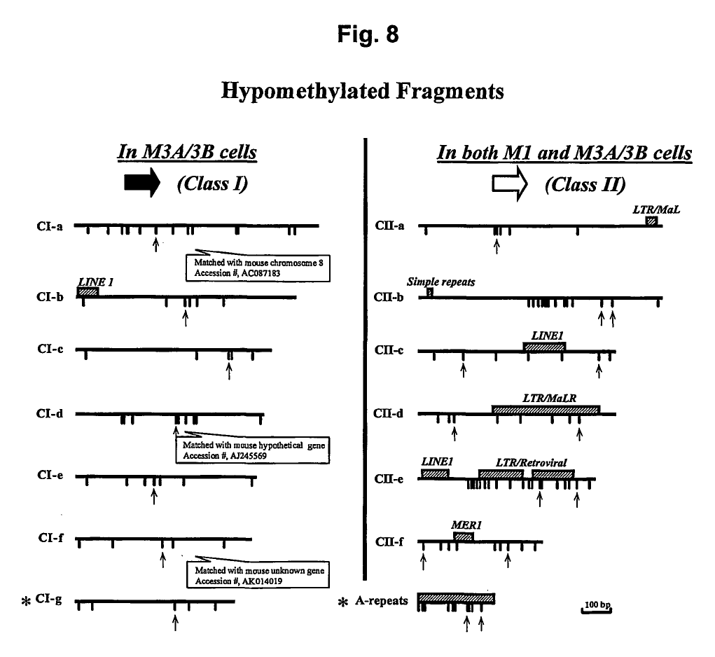

FIG. 8 shows the sequence properties of a selection of these two classes of fragments with respect to the occurrence of the CpG dinucleotides and the presence of repetitive elements. Class I sequences tended to have fewer repetitive elements, and a slightly lower CpG density than Class II sequences. Clearly, this distinction was not absolute since there were no obvious differences with respect to these properties such as the fragments CI-b and CII-a, or CI-a and CII-b.

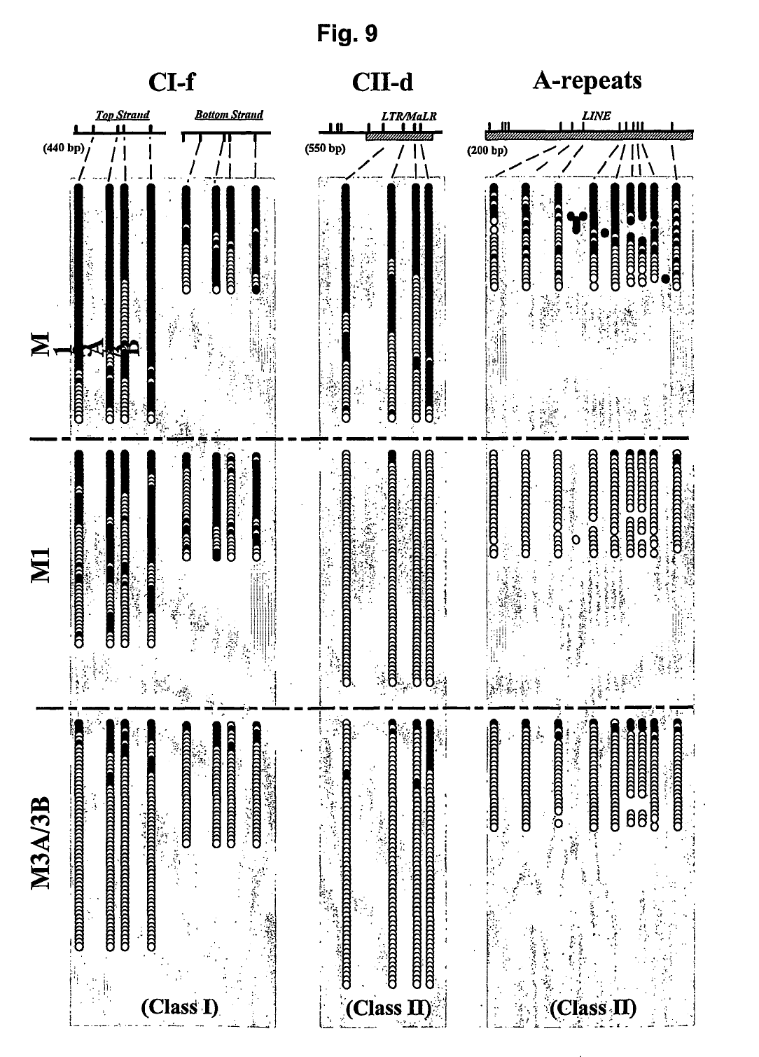

Methylation Patterns on Individual DNA Molecules. The analyses described above provided insight into the levels of methylation at individual CpG dinucleotides, but they did not yield information on the patterns of methylation at these regions. The sequencing of cloned PCR products of bisulfite-treated genomic DNA can reveal the patterns of methylation in individual DNA molecules. Therefore, such an analysis was performed on one of the Class I sequences, and two of the Class II sequences. These experiments clearly illustrated the differences in methylation levels in the different knockout cells (FIG. 9).

The 440-bp fragment CI-f sequence (GenBand Accession number AK014019) showed extensive methylation of all four sites sequenced in the wild-type cells and extensive methylation also in the M1 cells but a marked decrease in M3A/3B cells on both the top and bottom strands, which had approximately equal methylation levels at the CpG sites in all cell types. Analysis of the top strand of individual molecules in the M3A/3B cells showed a strong asymmetry of patterns in that 72% of molecules were completely unmethylated, 14% had only one site methylated, but 14% had multiple sites methylated on a single molecule. The differential levels of methylation at the four sites made it difficult to distinguish between the possibilities that the enzymes act processively, or rather that methylation at one site increases the probability of methylation of adjacent sites.

FIG. 9 also shows the dramatic decrease in the level of methylation of the CII-d fragment containing an LTR/MALR element in both the M1 and M3A/3B cells. There was substantial methylation in the wild-type cells but virtually no methylation in the M1 cells and low levels of methylation in M3A/3B cells. However the data clearly showed that there was little methylation of the four sites in this fragment in the M1 cells, in contrast to the CI-f fragment, which still showed substantial levels of methylation in M1 cells. A similar situation was also observed in the A-repeats (i.e., the promoters) of LINE-1 repetitive elements previously shown to be methylated in M3A/3B cells. Again, virtually no methylation occurred in M1 cells and slightly higher levels of methylation were seen in M3A/3B cells.

Since Dnmt1 is thought to be the major cytosine-S DNA methyltransferase in mammals, responsible for most, if not all maintenance activity in the cell, the identification of a class of sequences that appeared to be poorly maintained by Dnmt1 alone, was interesting. Dnmt3a or 3b did not seem to be independently responsible for the methylation of these sites, since these sequences were also substantially demethylated in M3A/3B cells (FIGS. 7 and 9). Furthermore, the effects of Dnmt1 and Dnmt3a/3b did not appear to be merely additive (FIG. 9), as would be the case if both Dnmt1 and Dnmt3a/3b were independent participants in the maintenance and de novo methylation of these sequences. Rather, methylation of these sequences appears to require cooperation both Dnmt1 and Dnmt 3a/3b activities. The most straightforward interpretation of these data is that Dnmt3a and/or 3b are necessary for ongoing de novo methylation of these sequences to compensate for poor maintenance methylation by Dnmt1. If either of these activities is absent, the methylation of these sequences cannot be properly maintained.

### EXAMPLE 7

### (A New Assay for the Detection of Hemi-methylation; Poor Maintenance Methylation was Balanced by a Continuing High Rate of de novo Methylation Mediated by Dnmt3a and/or Dnmt3b)

One of the sequences that was poorly maintained by Dnmt1 alone was further investigated, and showed that it had a surprisingly high level of hemi-methylation, even in wild-type cells, suggesting poor maintenance methylation balanced by a continuing high rate of de novo methylation mediated by Dnmt3a and/or Dnmt3b. This study used the inventive hemi-methylation assay, described herein above. Prior to the development of this novel and straightforward method there had been no accurate way to determine hemimethylation levels at specific CpG dinucleotides in the genome.

Further evidence that Dnmt3a and/or Dnmt3b are responsible for the compensating de novo methylation was provided by the fact that these enzymes could restore methylation to pretreatment levels following transient exposure of cells to 5-Aza-CdR, whereas Dnmt1 could not.

Dnmt1 by itself was also found herein to be incapable of restoring methylation of sequences that it had been able to maintain prior to the 5-Aza-CdR treatment, suggesting that its ability to de novo methylation is dependent on the presence of a critical level of pre-existing methylation at CpG sites.

A New Assay for the Detection of Hemi-methylation. One prediction of poor maintenance methylation of Class II sequences, offset by continuing de novo methylation is that the level of hemi-methylation at these sites (i.e., when only a single cytosine in the palindrome is methylated) should be substantially higher than in sequences with low levels of de novo methylation and very efficient maintenance methylation following each round of DNA replication.

Testing of this prediction required the development of a way to measure levels of hemi-methylation at individual CpG dinucleotides. The present invention provides such an invention as described herein under Examples 1-5 (see FIGS. 2 and 3). The assay was expanded to cover additional class I and class II genomic sequences.

FIG. 10 shows the results of the hemi-methylation analysis of a Class I sequence (CI-f) and of a Class II sequence (CII-d). There was a surprisingly high level of hemi-methylation of CII-d sequences in wild-type (M1/3A/3B) cells, consistent with the interpretation that the methylation of these sites is poorly maintained by Dnmt1, and requires ongoing de novo methylation mediated by Dnmt3a and/or Dnmt3b. Hemi-methylation levels of CI-f were substantially lower, as would be expected for a site that is subject to efficient maintenance methylation by Dnmt1. Control experiments of this sequences in M3A/3B cells demonstrated that equal amounts of hemi-methylation were present on top and bottom strands (FIG. 5).

Cooperativity Between Enzymes after 5-Aza-2 ′-deoxycytidine Treatment. Treatment of dividing cells with the DNA methyltransferase inhibitor 5-aza-CdR results in a transient demethylation of the genome, followed by a rebound of methylation levels in subsequent cell divisions. Since such rebound methylation requires de novo methylation activity, this type of analysis can provide an independent assessment of the de novo methylation capabilities of the various DNA methyltransferase enzymes at particular regions. This could lend further support to the proposition that Dnmt3a and/or Dnmt3b are responsible for ongoing de novo methylation of Class II sequences.

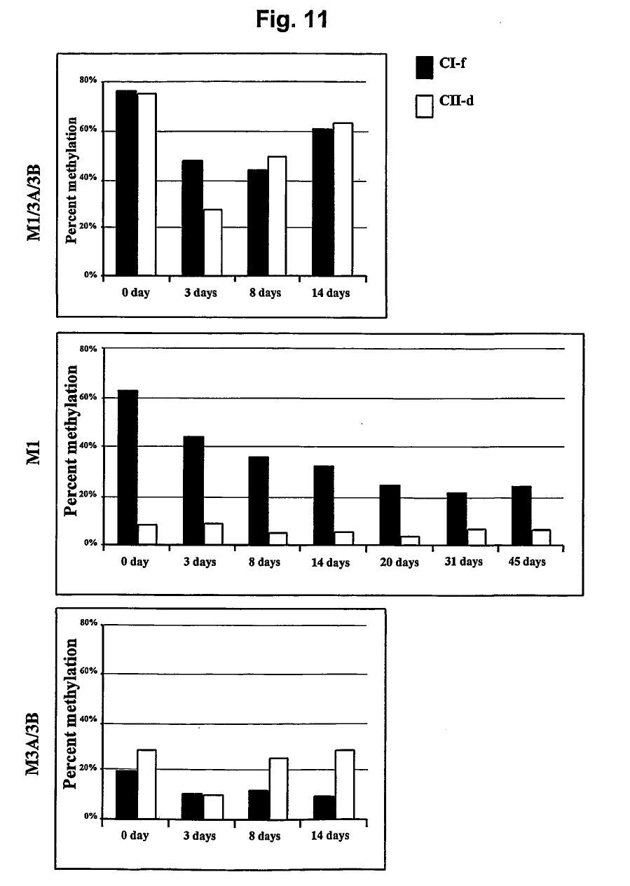

The knockout cells were, therefore, exposed to 5-aza-CdR and followed the kinetics of remethylation of the CI-f and CII-d fragments were followed to determine how the Dnmt's interacted to restore methylation levels after perturbation of the equilibrium in the ES cell types (FIG. 11). The results showed that methylation levels reached their minimum three days after drug treatment in wild type, and that the methylation of both fragments was restored to near pre-treatment levels by 14 days after treatment. FIG. 11 also shows that the combination of Dnmt3a with 3b (M3A/3B) was sufficient to result in the rapid remethylation of the CII-d but not the CI-f fragment.

Surprisingly, the Dnmt1 enzyme by itself failed to restore the methylation of either fragment after transient drug treatment (FIG. 11). The M1 cells showed a continued loss of methylation after treatment, suggesting that once the equilibrium had been perturbed below a certain level by 5-aza-CdR the Dnmt1 by itself or an as yet unidentified methyltransferase had insufficient de novo methylation activity for the restoration of methylation levels to pre-treatment levels. In contrast, the combined activities of Dnmt3a and Dnmt3b resulted in the full restoration of pretreatment levels of methylation at the CII-d sequence. These results suggest that the CII-d sequence is an efficient target for de novo methylation by Dnmt3a and/or Dnmt3b, but not by Dnmt1. This fits a model in which ongoing de novo methylating activity of Dnmt3a and/or 3b compensate for poor maintenance of this sequence by Dnmt1.

### EXAMPLE 8

### (Dnmt3a and/or Dnmt3b Methylation Occured Close to the Time of DNA Replication, While Dnmt1 Showed a Substantial Amount of Delayed Methylation)

Methylation by Dnmt3a and/or Dnmt3b occurs close to the time of DNA replication, while Dnmt1 shows a substantial amount of delayed methylation, extending beyond one hour post DNA synthesis. However, this delay in maintenance methylation by Dnmt1 was not responsible for the sequence-dependent variability in methylation levels in Dnmt3a/3b-deficient cells, since both types of sequences showed this maintenance methylation delay. We conclude that the major distinction between sites that are well maintained by Dnmt1, versus those that are not, lies in the efficiency of post-replicative maintenance methylation efficiency by Dnmt1, rather than in a difference in de novo methylation, or in delayed maintenance methylation.

Kinetics of Methylation of newly Synthesized DNA. The balance of poor maintenance methylation of Class II sequences, with a compensating de novo methylation by Dnmt3a and/or Dnmt3b raises the question whether both of these methylation activities occur concurrently immediately post replication, or whether the de novo methylation occurs at some other time in the cell cycle. A 1-hr BrdU pulse followed by chasing for various times was used to investigate the timing of methylation with respect to synthesis of the two DNA sequence classes.

DNA containing BrdU was isolated by immunoprecipitation using standard methods, and the methylation status of CpG sites within the fragments measured by cloning of individual DNA molecules and quantitation of methylation levels or by Ms-SNuPE analysis.

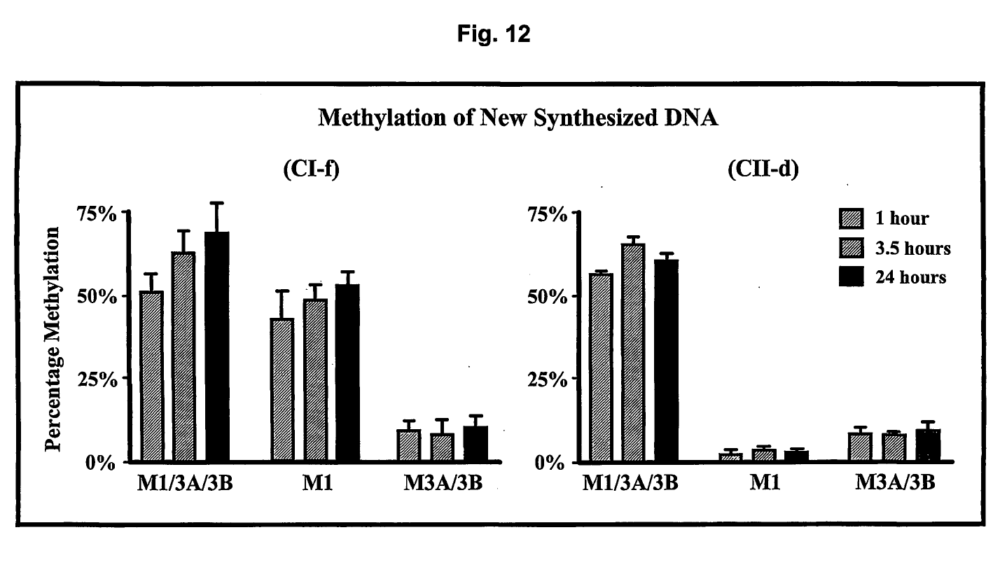

FIG. 12 summarizes results from such pulse-chase experiments which show that DNA synthesized during a 1-hr pulse of BrdU in wild-type (M1/3A/3B) cells was not methylated to its final level immediately after synthesis but that some further methylation occurred in the post-synthetic phase measured at 3.5 and 24 hr. However, this did not appear to be de novo methylation attributable to Dnmt3a and/or Dnmt3b, since M3A/3B cells did not show additional methylation after the first hour. Indeed, the delayed increase in methylation was also evident for the CI-f sequence in M1 cells. Prior demonstration that Dnmt1 is localized to the replication machinery in the S-phase nucleus and interacts with PCNA has led to the expectation in the art that the methylation pattern is copied immediately after synthesis. However, some earlier prior art studies have suggested that some methylation might be delayed, with cytosine methylation occurring in a biphasic fashion. The present results are consistent with these earlier studies.

In conclusion, most methylation by Dnmt3a and/or Dmnt3b occured immediately before or soon after DNA synthesis, while Dnmt1 continued to display maintenance activity more than 1 hour post replication.

### SUMMARY

### (EXAMPLES 1-8)

The functions of the three known DNA methyltransferases Dnmt1, Dnmt3a and 3b in establishing and maintaining methylation patterns are not fully understood. According to the present invention, mouse embryonic stem cells (ES) with systematic gene knockouts for Dmnt1, Dmnt3a and 3b were used to show that DNA methylation in a CpG-poor transcribed sequence occurs in two phases with respect to DNA synthesis. BrdU pulse chase experiments showed that Dnmt1 functioned at or soon after replication to methylate the newly synthesized strand to approximately 72% of the equilibrium level, but leaves patches of hemimethylated DNA. The restoration of the pattern was significantly facilitated by Dnmt3a and 3b which appear to be processive enzymes whose activities fill in the patches in addition to their roles as de novo methylases. The present invention provides novel methods to measure hemi-methylation and to calculate methylation rates. These novel methods were used herein to conclude that Dnmt3a and 3b act mainly as maintenance methyltransferases to ensure the completeness of methylation of the genome in ES cells.

The central and causative roles of DNA methylation in developmental processes such as imprinting and in pathological situations such as cancer, ICF and Rett syndromes are increasingly being appreciated. This has stimulated research to understand how methylation patterns are acquired and maintained. The recent cloning and preliminary characterization of the Dnmt3a and 3b enzymes has led to the suggestion that they might be primarily responsible for de novo methylation by establishing a pattern which is copied by the better characterized Dnmt1.

According to the present invention, Dnmt3a and 3b act as de novo enzymes, and likely have more important roles in non-differentiating cells may be to assist Dnmt1 in ensuring full maintenance of methylation patterns.

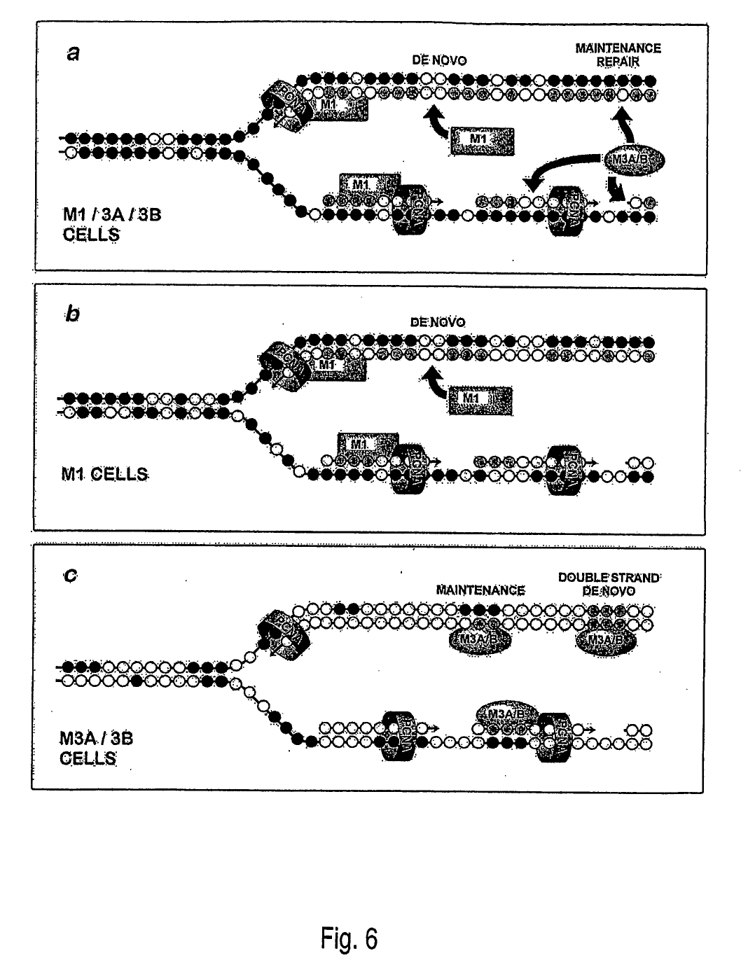

According to the present invention, the methylation of a region of DNA, typical of a mammalian gene with respect to the paucity of CpG, occurred in two phases and therefore discontinuously (summarized in the model shown in FIG. 6A, B and C).

FIGS. 6A, B and C illustrate the proposed model for the interactivity between DNA methyltransferase enzymes in ES cells. This figure represents a highly schematic interpretation of the data of the present invention. Although the data is based on the analysis of four contiguous CpGs, and of one CpG dinucleotide in particular detail, the conclusions have been summarized using a hypothetical replication fork in which evenly spaced CpG dinucleotides are represented by circles. Black circles indicate methylated CpGs on the parent DNA strands, while grey circles represent newly-methylated CpGs on the daughter strand. Unmethylated CpGs are indicated by white circles. PCNA is indicated by a ring. DNA methyltransferases are indicated by rectangles (Dnmt1) or ovals (Dnmt3a and/or 3b).

FIG. 6A depicts how methylation patterns are maintained in wild-type cells containing all three DNA methyltransferase enzymes. Most DNA synthesized in wild-type cells is likely immediately methylated after synthesis by Dnmt1 which is presumably closely associated with the replication machinery through its interaction with PCNA. There are regions of DNA, in which patches of hemimethylation occur, and these appear to serve as substrates for the Dnmt3a and 3b enzymes The full copying of the pattern requires the concerted efforts of all three enzymes.

FIG. 6B indicates how methylation is proposed to occur in cells lacking both Dmnt3a and Dnmt3b enzymes. Individual CpGs, or patches of CpGs missed by Dnmt1 remain unmethylated, since Dnmt3a and Dnmt3b are lacking. This results in a higher incidence of unmethylated CpGs surviving to the next round of replication. Some of these CpGs are methylated in the next round of replication by Dnmt1, so that by 24 hours, there are relatively few large patches of unmethylated CpGs in Ml cells (see FIG. 5).

FIG. 6C shows the situation in M3A/3B cells, which lack Dnmt1. A mosaic methylation pattern is present with patches of methylation and unmethylation and a substantial fraction of hemi-methylated sites. It is likely that in the absence of Dnmt1, Dnmt3a and/or 3b are capable of some copying of the existing methylation pattern, but are not particularly efficient. Furthermore, the enzymes also seem capable of a de novo modification of both strands of DNA, thus resulting in a mosaic pattern of methylation within these cell types.

The first phase of methylation, in which CpG sites are methylated to approximately 72% of their final levels, is most likely accomplished by Dmnt1, which has been shown to be associated with DNA replication machines by the elegant work of Leonhardt et al. (1992) and Rountree et al. (2000) and confirmed here.

Significantly, the enzyme left gaps of unmethylated sites linked to each other as shown by the direct sequencing of BrdU pulse labeled DNA. The size of these gaps or patches was not established, but they were at least 300 bp which is the distance between sites 1 and 4 in the fragment studied herein. This DNA contained a substantial number of hemimethylated sites, which were converted into fully methylated sites during the post synthetic phase. The delayed methylation was most likely accomplished by Dnmt3a/3b, which must therefore be acting as hemimethylases which seemed to be processive, yet not 100% efficient, enzymes.

The evidence for this processivity came from analysis of the M3A/3B cells which lacked Dnmt1 in which 72% of the molecules analyzed had no methylation whereas 14% had more than one site methylated. Putting these two properties together probably assures the stability of the pattern. However, it is also possible that the enzymes are not processive but rather that the presence of methylation attracts further methylation to the region.

Novel Hemi-methylation Assay. The present invention provides a novel method for the quantitation of hemi-methylation, which requires complete digestion of DNA with HpaII before bisulfite treatment to ensure accuracy. This was accomplished by the use of an excess of enzyme, and replenishment of enzyme after 16 hr incubation. The method is robust and has general utility in other experimental and applied applications.

The occurrence of a high percentage of hemimethylated sites in the M3A/3B cells seemed, at first sight, to not fit with the suggestion that the Dnmt3a and 3b enzymes acted as hemi-methylases to fill in gaps in wild-type cells. This apparent paradox could be explained if the overall level of cytosine methyltransferase activity in the M3A/3B cells was inadequate for effective methylation maintenance. This may indeed be the case since the combined rates of de novo and of maintenance methylation present in M3A/3B cells (0.07+0.37=0.44) was substantially lower than that of M1 cells (0.39+0.75=1.14) or of wild-type cells (0.50+0.94=1.44). Okano et al. (1998) found that Dnmt3a and Dnmt3b had substantially lower levels of activity than Dnmt1 in the test tube. The pattern in the M3A/3B cells is therefore highly mosaic with a significant percentage of hemimethylated patches which the Dnmt3a and 3b are unable to restore to a fully methylated configuration.

The overall rates described herein above were calculated from the measured steady-state levels of DNA methylation in cells with differing complements of DNA methylases. Nevertheless, the resulting rates appear to be mutually consistent, and suggest relatively little overlap in function. For instance, the combined de novo and maintenance methylation rates calculated for M1 cells (1.14) added to the combined de novo and maintenance methylation rates calculated for M3A/3B cells (0.44) equaled 1.58. This is not far from the calculated overall activity for M1/3A/3B cells of 1.44 suggesting that the activities are largely complementary rather than overlapping.

The equations of the present invention assume that all de novo methylation leads to a fully methylated product. Additionally, calculation methods were derived that consider de novo methyltransferase activity resulting in hemimethylation. However, the inclusion of such activities for purposes of the present invention leads to a less optimal fit with the experimental data. Additionally, experimental evidence from prokaryotic methyltransferases indicates that de novo methyltransferases act sequentially on the two opposing DNA strands, with the methylation of the cytosine on the first strand being rate-limiting.

The equations of the present invention assume that the top and bottom strands are equally methylated, a supposition supported in several preliminary experiments that were performed.

Calculations of the rates of hemimethylation, according to the present invention, assumes that the methylation state of a DNA strand reaches- the measured equilibrium level within a single cell division. In reality, the BrdU data suggested that, although DNA strands acquired most of their methylation within the first cell division, they continued to acquire additional methylation as they aged with each cell division.

Therefore, a pool of DNA molecules from a population of cells is comprised of many recently synthesized strands with DNA methylation levels at, or slightly below the average level for the entire population, as well as older DNA strands with higher levels of methylation. The relative prevalence of a DNA strand in the population decreases as a function of the number of rounds of DNA replication since its initial synthesis. These “strands” may represent long patches, rather than entire molecules (chromosomes), since recombination and repair-associated DNA synthesis could disrupt their contiguity.

The model, according to the present invention, supports a mechanism, whereby DNA strands continue to become increasingly methylated as they age. As a consequence, the rate calculations of the present invention should be viewed as weighted averages for the entire population of DNA strands of different age. Any de novo methylation that occurs on aging DNA strands could not be removed passively by DNA replication so that the only way to counteract this cumulative methylation would be to invoke active, enzymatic demethylation. Although these aging strands represent an increasingly small fraction of the population, they could have biological consequences if they reach a threshold of DNA methylation that results in a phenotypic effect, such silencing a tumor-suppressor gene, leading to clonal outgrowth. This previously unrecognized phenomenon may explain, in part, the increase in CpG island hypermethylation observed in aging (Ahuja and Issa, 2000) and in cancer (Jones and Laird, 1999).

In M1 cells lacking Dnmt3a and 3b, there was clear evidence for two classes of molecules with 50% of molecules newly synthesized during the 1-hr BrdU pulse having 2 or less sites concurrently methylated and the remainder containing 3 or 4 sites methylated (FIG. 5). The biphasic nature of the distribution was consistent with the idea that the leading and lagging strands were differentially handled by the methylases, although this has not been found to be the case with replication origins (Araujo et al., 1998). It was, however, interesting that the Dnmt3a and 3b enzymes showed differential nuclear localization, and further work will be necessary to determine whether the methylation kinetics of the leading and lagging strands differ.

The interpretation of data obtained according to the present invention relies to a significant extent on the use of BrdU to tag the newly synthesized DNA for subsequent analysis. It is therefore important to question whether the incorporation of this molecule into DNA might alter its post-synthetic methylation patterns. This is unlikely since early experiments showed directly that BrdU does not cause gross changes in methylation levels even at levels where 95% of thymines had been substituted by the analog (Singer et al., 1977).

Significantly, the methylation properties of a non-CpG island region of DNA was measured. Although most genomic methylation occurs in such sequences, it will be important to see whether the same cooperation between the enzymes is evident with CpG islands where modification is more tightly associated with promoter silencing. In this regard, it was determined that methylation of repetitive sequences was not well maintained in ES cells lacking Dnmt3a and 3b, indicating that at least two methyltransferases are needed to ensure methylation of these sequences, unlike the fragment studied herein which was substantially methylated in M1 cells. Also, replication origins in mammalian cells have been shown to become methylated within 5 min after synthesis (Araujo et al., 1998; Rein et al., 1999), suggesting that they may not show the delay apparent with the fragment we are studying.

The results of the present invention are strikingly reminiscent of complementary activities of other enzyme systems involved in DNA metabolism. For example, DNA polymerases α and δ participate at different stages of DNA synthesis (Waga and Stillman, 1998). Perhaps the methylation pattern is maintained by enzymes with overlapping yet specialized activities.

The data of the present invention shows that an important function of Dnmt3a and 3b in embryonic cells is to fill in patches of unmethylated DNA missed by Dnmt1 at the replication fork. These enzymes therefore have a repair function in addition to their potential roles as de novo methyltransferases.

Finally, Examples 6-8 show that mouse ES cells with systematic gene knockouts for DNA methyltransferases (Dnmts) can be used to delineate the roles of Dmnt1 and Dmnt3a and 3b in maintaining methylation patterns in the mouse genome. Dnmt1 alone was able to maintain methylation of most CpG-poor regions analyzed. In contrast, both Dnmt1 and Dnmt3a and/or Dnmt3b were required for the methylation of a select class of sequences which included abundant murine LINE-1 promoters.

A novel hemi-methylation assay was used to show that even in wildtype cells these sequences contain high levels of hemi-methylated DNA, suggestive of poor maintenance methylation. Dnmt3a and/or 3b could restore methylation of these sequences to pretreatment levels following transient exposure of cells to 5-Aza-CdR, whereas Dnmt1 by itself could not.

In conclusion, ongoing de novo methylation by Dnmt3a and/or Dmnt3b compensates for inefficient maintenance methylation by Dmnt1 of these endogenous repetitive sequences. The present results reveal a previously unrecognized degree of cooperativity among mammalian DNA methyltransferases in ES cells.

We claim:

1. A method to determine the fraction of DNA molecules that is hemi-methylated at a specific CpG dinucleotide sequence within a palindromic CpG methylation site, in a pool of DNA molecules having mixed DNA methylation states at that palindromic CpG site, comprising:
obtaining an isolated genomic DNA sample;
digesting the DNA sample with an excess of a methylation-sensitive restriction endonuclease that cleaves according to a recognition sequence motif comprising a palindromic CpG methylation site, whereby the endonuclease cleaves the DNA at a specific recognition motif position if the methylation state of the respective palindromic CpG methylation site is un-methylated, but not hemi-methylated or fully-methylated;
treating the endonuclease-digested DNA with bisulfite, whereby unmethylated cytosine residues on both strands of the DNA are converted to uracil;
amplifying one strand of the bisulfite-treated digested DNA with primers flanking the specific recognition motif position comprising a bisulfite-treated specific CpG dinucleotide of the palindromic CpG methylation site, whereby any uracil residues are converted to thymidine residues; and
determining, by sequence analysis of the amplification products or of individual clones thereof, the presence of either cytosine or thymidine at the position corresponding to the first nucleotide position of the specific CpG dinucleotide, whereby the fraction of DNA molecules that is hemi-methylated at the specific CpG dinucleotide is determined by averaging results obtained by the sequence analysis.

2. The method of claim 1, wherein the restriction endonculease is HpaII.

3. The method of claim 1, wherein amplifying the bisulfite-treated DNA occurs by PCR.

4. The method of claim 1, wherein sequence analysis is performed by a sequencing method selected from the group consisting of Ms-SNuPE, COBRA, DGE, MethyLight, MSP, MCA, direct sequencing, hybridization arrays, and combinations thereof.

5. The method of claim 1, further comprising digestion of the genomic DNA sample with a second restriction endonuclease.

6. The method of claim 5, wherein the second restriction endonuclease is RsaI.

7. A method to calculate at least one of the rate of de novo methylation (n), or the rate of maintenance methylation (m) at a specific CpG dinucleotide sequence within a palindromic CpG methylation site, comprising:
determining a value for S, where S is the percent of cytosines at a specific CpG dinucleotide found, by the method of claim 1, to be unmethylated by bisulfite analysis of one of the two single DNA strands;
determining a value for P, where P is the percent of cytosines at the specific CpG dinucleotide found, by the method of claim 1, to be methylated by-bisulfite analysis of one of the two DNA strands;
determining D, as D is 100-2S;
determining H, as H is 2SP/(S+D);
determining F, as F is DP/(S+D); and
determining U, as U is 100HF, whereby the rate of de novo methylation (n) at the specific CpG dinucleotide sequence is defined as: n=1−(U/(U+H/2)), where n is the fraction of unmethylated post-DNA synthesis specific CpG dinucleotide sequences that undergoes conversion to fully methylated CpG per cell division, and whereby the rate of maintenance methylation (m) at the specific CpG dinucleotide sequence is defined as: m=1−(H/(F+H/2)), where m is the fraction of post-DNA synthesis hemi-methylated specific CpG dinucleotide sequences that undergoes conversion to fully methylated CpG per cell division.

8. The method of claim 7, wherein determination of S and P are based on analysis of the same DNA strand.

9. A method to calculate the rate of maintenance methylation (m) at a specific CpG dinucleotide sequence within a palindromic CpG methylation site, comprising:
determining a value for S, where S is the percent of cytosines at a specific CpG dinucleotide found, by the method of claim 1, to be unmethylated by bisulfite analysis of one of the two single DNA strands;
determining a value for P, where P is the percent of cytosines at the specific CpG dinucleotide found, by the method of claim 1, to be methylated by bisulfite analysis of one of the two DNA strands;
determining D, as D is 100-2S;
determining H, as H is 2SP/(S+D); and
determining F, as F is DP/(S+D), whereby the rate of maintenance methylation (m) at the specific CpG dinucleotide sequence is defined as: m=1−(IV(F+H/2)), where m is the fraction of post-synthesis hemimethylated specific CpG dinucleotide sequences that undergoes conversion to fully methylated CpG per cell division.

10. The method of claim 9, wherein determination of S and P are based on analysis of the same DNA strand.

11. A methylation detection kit, in one or more containers, useful for measuring the fraction of DNA molecules that is hemi-methylated at a specific CpG dinucleotide in a pool of DNA with mixed DNA methylation states at that CpG, comprising:
a methylation-sensitive restriction endonuclease that cleaves according to a recognition sequence motif comprising a palindromic CpG methylation site, whereby the endonuclease cleaves the DNA at a specific recognition motif position if the methylation state of the respective palindromic CpG methylation site is un-methylated, but not hemi-methylated or fully-methylated; and
amplification primers flanking the specific recognition motif position.

12. The kit of claim 11, further comprising bisulfite treatment reagents.
